(19) United States
(12) Patent Application Publication
(10) Pub. No.: US 2021/0346507 A1
(43) Pub. Date: Nov. 11, 2021

(54) GEMCITABINE PRODRUGS

(71) Applicant: CURE BIOPHARMA INC., Carlsbad, CA (US)

(72) Inventors: Rajesh SHINDE, Lexington, MA (US); Laxman NALLAN, Rancho Mission Viejo, CA (US)

(21) Appl. No.: 17/269,978

(22) PCT Filed: Aug. 14, 2019
(86) PCT No.: PCT/US2019/046433

§ 371 Date: Feb. 19, 2021

Related U.S. Application Data
(60) Provisional application No. 62/719,729, filed on Aug. 20, 2018.

(51) Int. Cl. A61K 47/54; A61K 47/69; A61K 47/60; C07H 19/06

(52) U.S. Cl. CPC A61K 47/549; A61K 47/6929; A61K 47/6915; C07H 19/06; A61K 47/542; A61K 47/60; A61K 47/543

(57) ABSTRACT

Provided herein are phosphorylated gemcitabine derivative prodrug compounds, pharmaceutical compositions comprising said compounds, and methods for using said compounds for the treatment of cancer.

### BACKGROUND

A need exists in the medicinal arts for compositions and methods for gemcitabine prodrugs in the course of treating numerous types of cancer.

### BRIEF SUMMARY OF THE INVENTION

Provided herein are prodrugs of phosphorylated gemcitabine derivatives, pharmaceutical compositions comprising said compounds, and methods for using said compounds for the treatment of cancer.

One embodiment provides a compound, or a pharmaceutically acceptable salt thereof, having the structure of Formula (I):

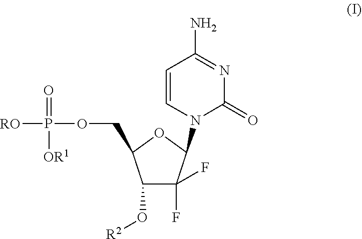

wherein

R is selected from fatty acid, glycerolipid, glycerophospholipids, sphingolipids, sterol-lipids, prenol lipids, saccharolipids and polyketides;

R¹ is selected from hydrogen, fatty acid, glycerolipid, glycerophospholipids, sphingolipids, sterol-lipids, prenol lipids, saccharolipids and polyketides; and

R² is selected from hydrogen, fatty acid, glycerolipid, glycerophospholipids, sphingolipids, sterol-lipids, prenol lipids, saccharolipids and polyketides.

Another embodiment provides a compound, or a pharmaceutically acceptable salt thereof, having the structure of Formula (II):

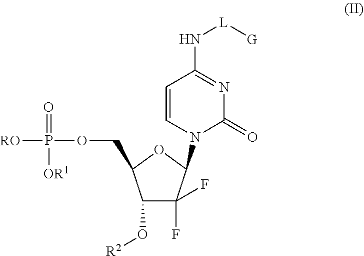

wherein

R is selected from hydrogen, fatty acid, glycerolipid, glycerophospholipids, sphingolipids, sterol-lipids, prenol lipids, saccharolipids and polyketides;

R¹ is selected from hydrogen, fatty acid, glycerolipid, glycerophospholipids, sphingolipids, sterol-lipids, prenol lipids, saccharolipids and polyketides;

R² is selected from hydrogen, fatty acid, glycerolipid, glycerophospholipids, sphingolipids, sterol-lipids, prenol lipids, saccharolipids and polyketides;

L is a linker selected from an alkylene amide group, an alkylene ester group, an alkylene carbamate group, a disulfide group, a phosphodiester group, and a phosphoramidate group; and

G is a cytotoxic chemotherapy agent.

Another embodiment provides a pharmaceutical composition comprising a compound of any one of Formula (I) or Formula (II), or pharmaceutically acceptable salt thereof, and a pharmaceutically acceptable excipient.

Another embodiment provides a method of treating cancer in a patient in need thereof, comprising administering to the patient a composition comprising a compound of any one of Formula (I) or Formula (II), or pharmaceutically acceptable salt thereof.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

Definitions

As used in the specification and appended claims, unless specified to the contrary, the following terms have the meaning indicated below.

“Amino” refers to the —NH₂ radical.

“Cyano” refers to the CN radical.

“Nitro” refers to the NO₂ radical.

“Oxa” refers to the —O— radical.

“Oxo” refers to the =O radical.

“Thioxo” refers to the =S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1propyl (n-propyl), 1-methylethyl (isopropyl), 1-butyl (nbutyl), 1-methylpropyl (sec-butyl), 2-methylpropyl (iso-butyl), 1,1 dimethylethyl (tertbutyl), 1pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), proplenyl (i.e., allyl), butlenyl, pentlenyl, pental, 4dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, N(R^{a})S(O)ₜR^{a} (where t is 1 or 2), S(O)ₜOR^{a} (where t is 1 or 2), S(O)ₜR^{a} (where t is 1 or 2) and S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, nbutylene, and the like. The alkylene chain is attached to the rest of the molecule through a single bond and to the radical group through a single bond. The points of attachment of the alkylene chain to the rest of the molecule and to the radical group is through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises two carbon atoms (e.g., C₂ alkenylene). In other embodiments, an alkenylene comprises five to eight carbon atoms (e.g., C₅-C₈ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkynylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2)π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}-OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula R^{c}aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula R^{e}aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —OR^{c}aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable nonaromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7dimethylbicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}carbocyclyl where R^{c} is an alkylene chain as defined above. The alkylene chain and the carbocyclyl radical is optionally substituted as defined above.

“Carbocyclylalkynyl” refers to a radical of the formula —R^{c}carbocyclyl where R^{c} is an alkynylene chain as defined above. The alkynylene chain and the carbocyclyl radical is optionally substituted as defined above. “Carbocyclylalkoxy” refers to a radical bonded through an oxygen atom of the formula —O—R^{c}carbocyclyl where R^{c} is an alkylene chain as defined above. The alkylene chain and the carbocyclyl radical is optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

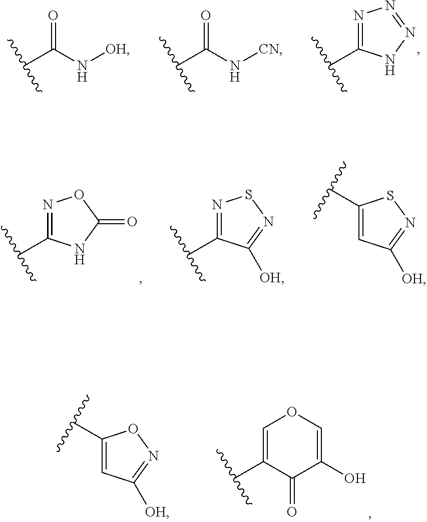

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2trifluoroethyl, 1fluoromethyl2fluoroethyl, and the like. In some embodiments, the alkyl part of the fluoroalkyl radical is optionally substituted as defined above for an alkyl group.

“Heterocyclyl” refers to a stable 3 to 18membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2oxopiperazinyl, 2oxopiperidinyl, 2oxopyrrolidinyl, oxazolidinyl, piperidinyl, piperazinyl, 4piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1oxothiomorpholinyl, and 1,1dioxothiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)-R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An Nheterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A Cheterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like. “Heterocyclylalkyl” refers to a radical of the formula —R^{c}heterocyclyl where R^{c} is an alkylene chain as defined above. If the heterocyclyl is a nitrogencontaining heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}heterocyclyl where R^{c} is an alkylene chain as defined above. If the heterocyclyl is a nitrogencontaining heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3 to 18membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1, 3benzodioxolyl, benzofuranyl, benzooxazolyl, benzo[d]thiazolyl, benzothiadiazolyl, benzo[b][1,4]dioxepinyl, benzo[b][1,4]oxazinyl, 1,4benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2d]pyrimidinyl, benzotriazolyl, benzo[4,6]imidazo[1,2a]pyridinyl, carbazolyl, cinnolinyl, cyclopenta[d]pyrimidinyl, 6,7dihydro5Hcyclopenta[4,5]thieno[2,3d]pyrimidinyl, 5, 6dihydrobenzo[h]quinazolinyl, 5,6dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2c]pyridinyl, 5,6,7,8,9,10hexahydrocycloocta[d]pyrimidinyl, 5,6,7,8,9,10hexahydrocycloocta[d]pyridazinyl, 5,6,7,8,9,10hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8methano5,6,7,8tetrahydroquinazolinyl, naphthyridinyl, 1,6naphthyridinonyl, oxadiazolyl, 2oxoazepinyl, oxazolyl, oxiranyl, 5, 6,6a,7,8,9,10,10aoctahydrobenzo[h]quinazolinyl, 1phenyl1Hpyrrolyl, phenazinyl, phenothiazinyl, phenoxazinyl, phthalazinyl, pteridinyl, purinyl, pyrrolyl, pyrazolyl, pyrazolo[3,4d]pyrimidinyl, pyridinyl, pyrido[3,2d]pyrimidinyl, pyrido[3,4d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7, 8tetrahydroquinazolinyl, 5,6,7,8tetrahydrobenzo[4,5]thieno[2,3d]pyrimidinyl, 6,7,8,9tetrahydro5Hcyclohepta[4,5]thieno[2,3d]pyrimidinyl, 5,6,7,8tetrahydropyrido[4,5c]pyridazinyl, thiazolyl, thiadiazolyl, triazolyl, tetrazolyl, triazinyl, thieno[2,3d]pyrimidinyl, thieno[3,2d]pyrimidinyl, thieno[2,3c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)-R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}heteroaryl, where R^{c} is an alkylene chain as defined above. If the heteroaryl is a nitrogencontaining heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}heteroaryl, where R^{c} is an alkylene chain as defined above. If the heteroaryl is a nitrogencontaining heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R) or (S). Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

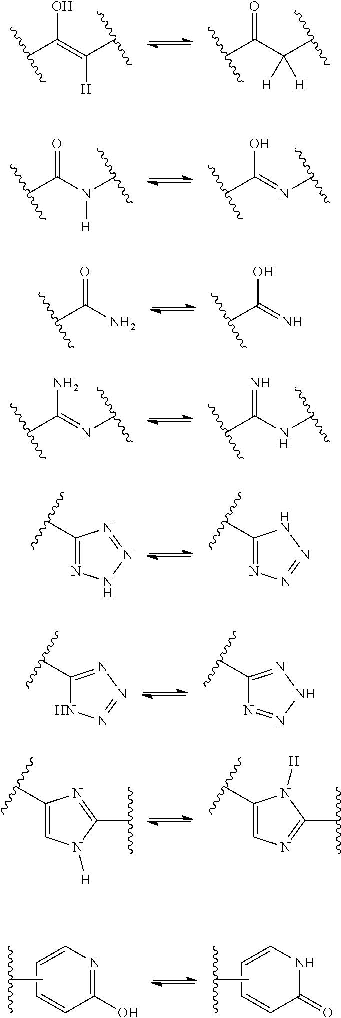

The compounds disclosed herein, in some embodiments, are used in different enriched isotopic forms, e.g., enriched in the content of ²H, ³H, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commerically from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

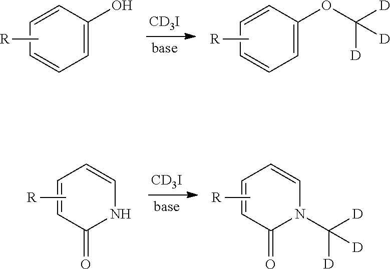

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

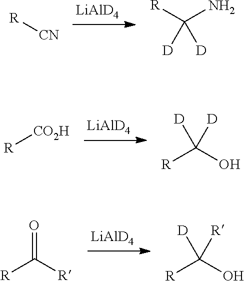

Deuterium gas and palladium catalyst are employed to reduce unsaturated carbon-carbon linkages and to perform a reductive substitution of aryl carbon-halogen bonds as illustrated, by way of example only, in the reaction schemes below.

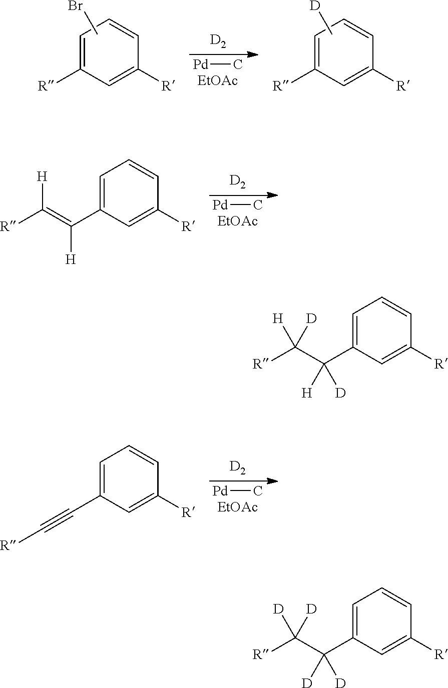

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the gemcitabine prodrug compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, ptoluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2dimethylaminoethanol, 2diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, Nethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

Phosphorylated Gemcitabine Derivatives

A number of nucleoside analogs such as cytarabine, fludarabine, cladribine, capecitabine, gemcitabine and pentostatin are used clinically as highly effective anti-neoplastic agents. Among these, gemcitabine (2′,2′-difluoro-2′-deoxycytidine) is of particular interest due to its unique activity against solid tumors. It is currently approved to treat breast, non-small cell lung, ovarian and pancreatic cancers and widely used to treat a variety of other cancers including bladder, biliary, colorectal and lymphoma.

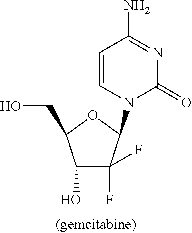 [SMILES: Nc1ccn([C@@H]2O[C@H](CO)[C@@H](O)C2(F)F)c(=O)n1]

Several self-potentiating mechanisms unique to this nucleoside analog are believed responsible for the activity of gemcitabine against solid tumors. The diphosphate metabolite of gemcitabine inhibits ribonucleotide reductase, which results in lower concentrations of intracellular deoxycytidine triphosphate (dCTP) and thus, increased incorporation of the triphosphate gemcitabine metabolite into DNA, which results in inhibition of DNA synthesis and blocks completion of the cell division cycle. Additionally, reduction in dCTP concentration upregulates the enzyme cytidine kinase, which is responsible for initial phosphorylation of gemcitabine, a necessary step in the inhibition of DNA synthesis by the drug. Finally, the triphosphate metabolite of gemcitabine is an inhibitor of cytidine deaminase, which is responsible for gemcitabine inactivation by conversion to the uridine metabolite. Accordingly, the additive nature of the above factors may explain the efficacy of gemcitabine in treating solid tumors.

Previous studies have characterized multiple cellular transport mechanisms for nucleoside analog drugs and their derivatives (for a review, see Balimane et al., Adv. Drug Delivery Rev. 1999, 39, 183-209). A relatively hydrophilic compound, gemcitabine has limited ability to permeate plasma membranes via passive diffusion and several studies have demonstrated that gemcitabine is a substrate for equilibrative and concentrative nucleoside transporters (ENT's and CNT's respectively). Specifically, gemcitabine is transported by human ENT1, ENT2, CNT1 and CNT3, but not the purine-selective concentrative transporter CNT2 (see Mackey et al., Cancer Res. 1998, 58, 4349-4357; Mackey et al., J. Natl. Cancer Inst. 1999, 91, 1876-1881; and Fang et al., Biochem. J. 1996, 317, 457465).

Gemcitabine is itself a prodrug that is activated in a series of phosphorylation steps to its activated form of 5′-triphosphate. Typically, once gemcitabine enters the tumor cell, it undergoes a series of phosphorylations, which are generally believed to make it active. First, it is generally phosphorylated to a monophosphate compound (dFdCMP) by deoxycytidine kinase (dCK). It then often undergoes a second modification to become gemcitabine diphosphate (dFdCDP), and finally can be converted to gemcitabine triphosphate (dFdCTP). These forms can be catalyzed by the enzymes nucleoside monophosphate kinase (UMP/CMP) and diphosphate kinase, respectively. Gemcitabine can be inactivated by cytidine deaminase (CDA). The deamination of gemcitabine monophosphate, which is catalysed by deoxycytidylate deaminase (DCTD), also deactivates the drug. Phosphorylated metabolites of gemcitabine can be reduced by cellular 5′-nucleotidase (5′-NT), and dFdCMP can also be converted and inactivated by DCTD into 2′-deoxy-2′,2′-difluorouridine monophosphate (dFdUMP). Resistance to gemcitabine comes from at least 3 potential pathways: 1) deficiency of hENT pathway that helps dFdC cross into cells, 2) down regulation of cDK involved in phosphorylation and/or upregulation of cytidine deaminase (CDA) responsible for deamination of dFdC to 2′,2′-difluorodeoxyuridine (dFdU), of which the activity is uncertain, and 3) high efflux of drug by transporters such as ABC transporters. Accordingly, there is a need for gemcitabine analogs, which are not susceptible to the various resistance pathways while retaining the efficacious anti-tumor activity of the parent drug. One of the solutions to overcome the problem of gemcitabine resistance is to use phosphorylated gemcitabine prodrug compounds which provide a long, sustained, and controlled release of the biologically active phosphorylated gemcitabine derivatives upon administration into the body. Administration of the phosphorylated gemcitabine derivatives may provide a more convenient dosing regimen, greater efficacy, and reduce side effects in comparison with a traditional intravenous infusion of gemcitabine as typically used in cancer therapy.

As used in this disclosure, the term “prodrug” is meant to indicate a compound that is converted under physiological conditions to gemcitabine, or the phosphorylated forms thereof. A prodrug, in some embodiments, is inactive when administered to a subject, but is converted in vivo to an active compound, for example, by hydrolysis. Thus, the term “prodrug” refers to a precursor compound that is pharmaceutically acceptable, and in some embodiments, is devoid of the pharmacological properties of gemcitabine. The prodrug compound often offers advantages of solubility, tissue compatibility or delayed release in a mammalian organism (see, e.g., Bundgard, H., Design of Prodrugs (1985), pp. 79, 2124 (Elsevier, Amsterdam).

A discussion of prodrugs is provided in Higuchi, T., et al., “Prodrugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” is also meant to include any covalently bonded carriers that release the active compound in vivo when such prodrug is administered to a mammalian subject. Prodrugs of gemcitabine, as described herein, are prepared by modifying functional groups present in the active compound in such a way that the modifications are cleaved to the parent active compound. Prodrugs include compounds wherein a hydroxy group is bonded to any group that, when the prodrug of the active compound is administered to a mammalian subject, cleaves to form a free hydroxy group.

Provided herein are phosphorylated gemcitabine derivative prodrugs. The prodrugs described herein offer several exemplary advantages. First, the prodrugs generally comprise a modified mono-phosphate form of gemcitabine. Use of a mono-phosphate form of gemcitabine may allow the prodrugs to be converted to the active forms of the compound without undergoing the phosphorylation events described above. Thus, the prodrug might be less susceptible to certain forms of resistance that can develop when a subject becomes unable to convert gemcitabine to the phosphorylated active forms.

Second, the prodrugs described herein are often engineered to be lipophilic. Lipophilic gemcitabine derivative prodrugs may exhibit increased uptake by cancer cells, exhibit advantageous toxicity profiles, or provide enhance the therapeutic windows compared to gemcitabine. Lipophilic gemcitabine derivative prodrugs may also bypass traditional pathways to enter a cell, modulate distribution of the drug in the body, and allow for alternative delivery mechanisms and formulations as comparied to gemcitabine. In one aspect, provided herein is a compound, or pharmaceutically acceptable salt thereof, having a structure provided in Formula (I),

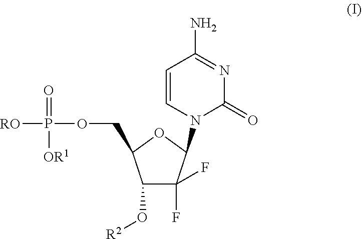

wherein

R is selected from fatty acid, glycerolipid, glycerophospholipids, sphingolipids, sterol-lipids, prenol lipids, saccharolipids and polyketides;

R¹ is selected from hydrogen, fatty acid, glycerolipid, glycerophospholipids, sphingolipids, sterol-lipids, prenol lipids, saccharolipids and polyketides; and

R² is selected from hydrogen, fatty acid, glycerolipid, glycerophospholipids, sphingolipids, sterol-lipids, prenol lipids, saccharolipids and polyketides.

In some embodiments, R¹ is hydrogen. In some embodiments, R² is hydrogen. In some embodiments, R¹ and R² are hydrogen.

In some embodiments, R¹ is not hydrogen. In some embodiments, R¹ is not hydrogen, and R and R¹ are selected independently. In some embodiments, R¹ is not hydrogen, and R and R¹ are the same. In some embodiments, R¹ is not hydrogen and R² is hydrogen.

In some embodiments, R² is not hydrogen. In some embodiments, R² is not hydrogen, and R and R² are selected independently. In some embodiments, R² is not hydrogen, and R and R² are the same. In some embodiments, R¹ is hydrogen and R² is not hydrogen.

In some embodiments, R and R¹ are independently selected from a fatty acid. In some embodiments, R is a fatty acid, and R¹ is hydrogen. In some embodiments, R and R² are independently selected from a fatty acid. In some embodiments, R is a fatty acid, and R² is hydrogen.

In some embodiments, R¹ and R² are both hydrogen.

In some embodiments, each fatty acid is independently selected from a saturated, monounsaturated, or polyunsaturated fatty acid.

In some embodiments, each fatty acid is a C2-C26 fatty acid. In some embodiments, each fatty acid is a C2-C3 fatty acid. In some embodiments, each fatty acid is a C2-C4 fatty acid. In some embodiments, each fatty acid is a C2-C5 fatty acid. In some embodiments, each fatty acid is a C2-C6 fatty acid. In some embodiments, each fatty acid is a C2-C7 fatty acid. In some embodiments, each fatty acid is a C2-C8 fatty acid. In some embodiments, each fatty acid is a C2-C9 fatty acid. In some embodiments, each fatty acid is a C2-C10 fatty acid. In some embodiments, each fatty acid is a C2-C11 fatty acid. In some embodiments, each fatty acid is a C2-C12 fatty acid. In some embodiments, each fatty acid is a C2-C13 fatty acid. In some embodiments, each fatty acid is a C2-C14 fatty acid. In some embodiments, each fatty acid is a C2-C15 fatty acid. In some embodiments, each fatty acid is a C2-C16 fatty acid. In some embodiments, each fatty acid is a C2-C17 fatty acid. In some embodiments, each fatty acid is a C2-C18 fatty acid. In some embodiments, each fatty acid is a C2-C19 fatty acid. In some embodiments, each fatty acid is a C2-C20 fatty acid. In some embodiments, each fatty acid is a C2-C21 fatty acid. In some embodiments, each fatty acid is a C2-C22 fatty acid. In some embodiments, each fatty acid is a C2-C23 fatty acid. In some embodiments, each fatty acid is a C2-C24 fatty acid. In some embodiments, each fatty acid is a C2-C25 fatty acid. In some embodiments, each fatty acid is a C2-C26 fatty acid. In some embodiments, each fatty acid is a C3-C4 fatty acid. In some embodiments, each fatty acid is a C3-C5 fatty acid. In some embodiments, each fatty acid is a C3-C6 fatty acid. In some embodiments, each fatty acid is a C3-C7 fatty acid. In some embodiments, each fatty acid is a C3-C8 fatty acid. In some embodiments, each fatty acid is a C3-C9 fatty acid. In some embodiments, each fatty acid is a C3-C10 fatty acid. In some embodiments, each fatty acid is a C3-C11 fatty acid. In some embodiments, each fatty acid is a C3-C12 fatty acid. In some embodiments, each fatty acid is a C3-C13 fatty acid. In some embodiments, each fatty acid is a C3-C14 fatty acid. In some embodiments, each fatty acid is a C3-C15 fatty acid. In some embodiments, each fatty acid is a C3-C16 fatty acid. In some embodiments, each fatty acid is a C3-C17 fatty acid. In some embodiments, each fatty acid is a C3-C18 fatty acid. In some embodiments, each fatty acid is a C3-C19 fatty acid. In some embodiments, each fatty acid is a C3-C20 fatty acid. In some embodiments, each fatty acid is a C3-C21 fatty acid. In some embodiments, each fatty acid is a C3-C22 fatty acid. In some embodiments, each fatty acid is a C3-C23 fatty acid. In some embodiments, each fatty acid is a C3-C24 fatty acid. In some embodiments, each fatty acid is a C3-C25 fatty acid. In some embodiments, each fatty acid is a C3-C26 fatty acid. In some embodiments, each fatty acid is a C4-C5 fatty acid. In some embodiments, each fatty acid is a C4-C6 fatty acid. In some embodiments, each fatty acid is a C4-C7 fatty acid. In some embodiments, each fatty acid is a C4-C8 fatty acid. In some embodiments, each fatty acid is a C4-C9 fatty acid. In some embodiments, each fatty acid is a C4-C10 fatty acid. In some embodiments, each fatty acid is a C4-C11 fatty acid. In some embodiments, each fatty acid is a C4-C12 fatty acid. In some embodiments, each fatty acid is a C4-C13 fatty acid. In some embodiments, each fatty acid is a C4-C14 fatty acid. In some embodiments, each fatty acid is a C4-C15 fatty acid. In some embodiments, each fatty acid is a C4-C16 fatty acid. In some embodiments, each fatty acid is a C4-C17 fatty acid. In some embodiments, each fatty acid is a C4-C18 fatty acid. In some embodiments, each fatty acid is a C4-C19 fatty acid. In some embodiments, each fatty acid is a C4-C20 fatty acid. In some embodiments, each fatty acid is a C4-C21 fatty acid. In some embodiments, each fatty acid is a C4-C22 fatty acid. In some embodiments, each fatty acid is a C4-C23 fatty acid. In some embodiments, each fatty acid is a C4-C24 fatty acid. In some embodiments, each fatty acid is a C4-C25 fatty acid. In some embodiments, each fatty acid is a C4-C26 fatty acid. In some embodiments, each fatty acid is a C5-C6 fatty acid. In some embodiments, each fatty acid is a C5-C7 fatty acid. In some embodiments, each fatty acid is a C5-C8 fatty acid. In some embodiments, each fatty acid is a C5-C9 fatty acid. In some embodiments, each fatty acid is a C5-C10 fatty acid. In some embodiments, each fatty acid is a C5-C11 fatty acid. In some embodiments, each fatty acid is a C5-C12 fatty acid. In some embodiments, each fatty acid is a C5-C13 fatty acid. In some embodiments, each fatty acid is a C5-C14 fatty acid. In some embodiments, each fatty acid is a C5-C15 fatty acid. In some embodiments, each fatty acid is a C5-C16 fatty acid. In some embodiments, each fatty acid is a C5-C17 fatty acid. In some embodiments, each fatty acid is a C5-C18 fatty acid. In some embodiments, each fatty acid is a C5-C19 fatty acid. In some embodiments, each fatty acid is a C5-C20 fatty acid. In some embodiments, each fatty acid is a C5-C21 fatty acid. In some embodiments, each fatty acid is a C5-C22 fatty acid. In some embodiments, each fatty acid is a C5-C23 fatty acid. In some embodiments, each fatty acid is a C5-C24 fatty acid. In some embodiments, each fatty acid is a C5-C25 fatty acid. In some embodiments, each fatty acid is a C5-C26 fatty acid. In some embodiments, each fatty acid is a C6-C7 fatty acid. In some embodiments, each fatty acid is a C6-C8 fatty acid. In some embodiments, each fatty acid is a C6-C9 fatty acid. In some embodiments, each fatty acid is a C6-C10 fatty acid. In some embodiments, each fatty acid is a C6-C11 fatty acid. In some embodiments, each fatty acid is a C6-C12 fatty acid. In some embodiments, each fatty acid is a C6-C13 fatty acid. In some embodiments, each fatty acid is a C6-C14 fatty acid. In some embodiments, each fatty acid is a C6-C15 fatty acid. In some embodiments, each fatty acid is a C6-C16 fatty acid. In some embodiments, each fatty acid is a C6-C17 fatty acid. In some embodiments, each fatty acid is a C6-C18 fatty acid. In some embodiments, each fatty acid is a C6-C19 fatty acid. In some embodiments, each fatty acid is a C6-C20 fatty acid. In some embodiments, each fatty acid is a C6-C21 fatty acid. In some embodiments, each fatty acid is a C6-C22 fatty acid. In some embodiments, each fatty acid is a C6-C23 fatty acid. In some embodiments, each fatty acid is a C6-C24 fatty acid. In some embodiments, each fatty acid is a C6-C25 fatty acid. In some embodiments, each fatty acid is a C6-C26 fatty acid. In some embodiments, each fatty acid is a C7-C8 fatty acid. In some embodiments, each fatty acid is a C7-C9 fatty acid. In some embodiments, each fatty acid is a C7-C10 fatty acid. In some embodiments, each fatty acid is a C7-C11 fatty acid. In some embodiments, each fatty acid is a C7-C12 fatty acid. In some embodiments, each fatty acid is a C7-C13 fatty acid. In some embodiments, each fatty acid is a C7-C14 fatty acid. In some embodiments, each fatty acid is a C7-C15 fatty acid. In some embodiments, each fatty acid is a C7-C16 fatty acid. In some embodiments, each fatty acid is a C7-C17 fatty acid. In some embodiments, each fatty acid is a C7-C18 fatty acid. In some embodiments, each fatty acid is a C7-C19 fatty acid. In some embodiments, each fatty acid is a C7-C20 fatty acid. In some embodiments, each fatty acid is a C7-C21 fatty acid. In some embodiments, each fatty acid is a C7-C22 fatty acid. In some embodiments, each fatty acid is a C7-C23 fatty acid. In some embodiments, each fatty acid is a C7-C24 fatty acid. In some embodiments, each fatty acid is a C7-C25 fatty acid. In some embodiments, each fatty acid is a C7-C26 fatty acid. In some embodiments, each fatty acid is a C8-C9 fatty acid. In some embodiments, each fatty acid is a C8-C10 fatty acid. In some embodiments, each fatty acid is a C8-C11 fatty acid. In some embodiments, each fatty acid is a C8-C12 fatty acid. In some embodiments, each fatty acid is a C8-C13 fatty acid. In some embodiments, each fatty acid is a C8-C14 fatty acid. In some embodiments, each fatty acid is a C8-C15 fatty acid. In some embodiments, each fatty acid is a C8-C16 fatty acid. In some embodiments, each fatty acid is a C8-C17 fatty acid. In some embodiments, each fatty acid is a C8-C18 fatty acid. In some embodiments, each fatty acid is a C8-C19 fatty acid. In some embodiments, each fatty acid is a C8-C20 fatty acid. In some embodiments, each fatty acid is a C8-C21 fatty acid. In some embodiments, each fatty acid is a C8-C22 fatty acid. In some embodiments, each fatty acid is a C8-C23 fatty acid. In some embodiments, each fatty acid is a C8-C24 fatty acid. In some embodiments, each fatty acid is a C8-C25 fatty acid. In some embodiments, each fatty acid is a C8-C26 fatty acid. In some embodiments, each fatty acid is a C9-C10 fatty acid. In some embodiments, each fatty acid is a C9-C11 fatty acid. In some embodiments, each fatty acid is a C9-C12 fatty acid. In some embodiments, each fatty acid is a C9-C13 fatty acid. In some embodiments, each fatty acid is a C9-C14 fatty acid. In some embodiments, each fatty acid is a C9-C15 fatty acid. In some embodiments, each fatty acid is a C9-C16 fatty acid. In some embodiments, each fatty acid is a C9-C17 fatty acid. In some embodiments, each fatty acid is a C9-C18 fatty acid. In some embodiments, each fatty acid is a C9-C19 fatty acid. In some embodiments, each fatty acid is a C9-C20 fatty acid. In some embodiments, each fatty acid is a C9-C21 fatty acid. In some embodiments, each fatty acid is a C9-C22 fatty acid. In some embodiments, each fatty acid is a C9-C23 fatty acid. In some embodiments, each fatty acid is a C9-C24 fatty acid. In some embodiments, each fatty acid is a C9-C25 fatty acid. In some embodiments, each fatty acid is a C9-C26 fatty acid. In some embodiments, each fatty acid is a C10-C11 fatty acid. In some embodiments, each fatty acid is a C10-C12 fatty acid. In some embodiments, each fatty acid is a C10-C13 fatty acid. In some embodiments, each fatty acid is a C10-C14 fatty acid. In some embodiments, each fatty acid is a C10-C15 fatty acid. In some embodiments, each fatty acid is a C10-C16 fatty acid. In some embodiments, each fatty acid is a C10-C17 fatty acid. In some embodiments, each fatty acid is a C10-C18 fatty acid. In some embodiments, each fatty acid is a C10-C19 fatty acid. In some embodiments, each fatty acid is a C10-C20 fatty acid. In some embodiments, each fatty acid is a C10-C21 fatty acid. In some embodiments, each fatty acid is a C10-C22 fatty acid. In some embodiments, each fatty acid is a C10-C23 fatty acid. In some embodiments, each fatty acid is a C10-C24 fatty acid. In some embodiments, each fatty acid is a C10-C25 fatty acid. In some embodiments, each fatty acid is a C10-C26 fatty acid. In some embodiments, each fatty acid is a C11-C12 fatty acid. In some embodiments, each fatty acid is a C11-C13 fatty acid. In some embodiments, each fatty acid is a C11-C14 fatty acid. In some embodiments, each fatty acid is a C11-C15 fatty acid. In some embodiments, each fatty acid is a C11-C16 fatty acid. In some embodiments, each fatty acid is a C11-C17 fatty acid. In some embodiments, each fatty acid is a C11-C18 fatty acid. In some embodiments, each fatty acid is a C11-C19 fatty acid. In some embodiments, each fatty acid is a C11-C20 fatty acid. In some embodiments, each fatty acid is a C11-C21 fatty acid. In some embodiments, each fatty acid is a C11-C22 fatty acid. In some embodiments, each fatty acid is a C11-C23 fatty acid. In some embodiments, each fatty acid is a C11-C24 fatty acid. In some embodiments, each fatty acid is a C11-C25 fatty acid. In some embodiments, each fatty acid is a C11-C26 fatty acid. In some embodiments, each fatty acid is a C12-C13 fatty acid. In some embodiments, each fatty acid is a C12-C14 fatty acid. In some embodiments, each fatty acid is a C12-C15 fatty acid. In some embodiments, each fatty acid is a C12-C16 fatty acid. In some embodiments, each fatty acid is a C12-C17 fatty acid. In some embodiments, each fatty acid is a C12-C18 fatty acid. In some embodiments, each fatty acid is a C12-C19 fatty acid. In some embodiments, each fatty acid is a C12-C20 fatty acid. In some embodiments, each fatty acid is a C12-C21 fatty acid. In some embodiments, each fatty acid is a C12-C22 fatty acid. In some embodiments, each fatty acid is a C12-C23 fatty acid. In some embodiments, each fatty acid is a C12-C24 fatty acid. In some embodiments, each fatty acid is a C12-C25 fatty acid. In some embodiments, each fatty acid is a C12-C26 fatty acid. In some embodiments, each fatty acid is a C13-C14 fatty acid. In some embodiments, each fatty acid is a C13-C15 fatty acid. In some embodiments, each fatty acid is a C13-C16 fatty acid. In some embodiments, each fatty acid is a C13-C17 fatty acid. In some embodiments, each fatty acid is a C13-C18 fatty acid. In some embodiments, each fatty acid is a C13-C19 fatty acid. In some embodiments, each fatty acid is a C13-C20 fatty acid. In some embodiments, each fatty acid is a C13-C21 fatty acid. In some embodiments, each fatty acid is a C13-C22 fatty acid. In some embodiments, each fatty acid is a C13-C23 fatty acid. In some embodiments, each fatty acid is a C13-C24 fatty acid. In some embodiments, each fatty acid is a C13-C25 fatty acid. In some embodiments, each fatty acid is a C13-C26 fatty acid. In some embodiments, each fatty acid is a C14-C15 fatty acid. In some embodiments, each fatty acid is a C14-C16 fatty acid. In some embodiments, each fatty acid is a C14-C17 fatty acid. In some embodiments, each fatty acid is a C14-C18 fatty acid. In some embodiments, each fatty acid is a C14-C19 fatty acid. In some embodiments, each fatty acid is a C14-C20 fatty acid. In some embodiments, each fatty acid is a C14-C21 fatty acid. In some embodiments, each fatty acid is a C14-C22 fatty acid. In some embodiments, each fatty acid is a C14-C23 fatty acid. In some embodiments, each fatty acid is a C14-C24 fatty acid. In some embodiments, each fatty acid is a C14-C25 fatty acid. In some embodiments, each fatty acid is a C14-C26 fatty acid. In some embodiments, each fatty acid is a C15-C16 fatty acid. In some embodiments, each fatty acid is a C15-C17 fatty acid. In some embodiments, each fatty acid is a C15-C18 fatty acid. In some embodiments, each fatty acid is a C15-C19 fatty acid. In some embodiments, each fatty acid is a C15-C20 fatty acid. In some embodiments, each fatty acid is a C15-C21 fatty acid. In some embodiments, each fatty acid is a C15-C22 fatty acid. In some embodiments, each fatty acid is a C15-C23 fatty acid. In some embodiments, each fatty acid is a C15-C24 fatty acid. In some embodiments, each fatty acid is a C15-C25 fatty acid. In some embodiments, each fatty acid is a C15-C26 fatty acid. In some embodiments, each fatty acid is a C16-C17 fatty acid. In some embodiments, each fatty acid is a C16-C18 fatty acid. In some embodiments, each fatty acid is a C16-C19 fatty acid. In some embodiments, each fatty acid is a C16-C20 fatty acid. In some embodiments, each fatty acid is a C16-C21 fatty acid. In some embodiments, each fatty acid is a C16-C22 fatty acid. In some embodiments, each fatty acid is a C16-C23 fatty acid. In some embodiments, each fatty acid is a C16-C24 fatty acid. In some embodiments, each fatty acid is a C16-C25 fatty acid. In some embodiments, each fatty acid is a C16-C26 fatty acid. In some embodiments, each fatty acid is a C17-C18 fatty acid. In some embodiments, each fatty acid is a C17-C19 fatty acid. In some embodiments, each fatty acid is a C17-C20 fatty acid. In some embodiments, each fatty acid is a C17-C21 fatty acid. In some embodiments, each fatty acid is a C17-C22 fatty acid. In some embodiments, each fatty acid is a C17-C23 fatty acid. In some embodiments, each fatty acid is a C17-C24 fatty acid. In some embodiments, each fatty acid is a C17-C25 fatty acid. In some embodiments, each fatty acid is a C17-C26 fatty acid. In some embodiments, each fatty acid is a C18-C19 fatty acid. In some embodiments, each fatty acid is a C18-C20 fatty acid. In some embodiments, each fatty acid is a C18-C21 fatty acid. In some embodiments, each fatty acid is a C18-C22 fatty acid. In some embodiments, each fatty acid is a C18-C23 fatty acid. In some embodiments, each fatty acid is a C18-C24 fatty acid. In some embodiments, each fatty acid is a C18-C25 fatty acid. In some embodiments, each fatty acid is a C18-C26 fatty acid. In some embodiments, each fatty acid is a C19-C20 fatty acid. In some embodiments, each fatty acid is a C19-C21 fatty acid. In some embodiments, each fatty acid is a C19-C22 fatty acid. In some embodiments, each fatty acid is a C19-C23 fatty acid. In some embodiments, each fatty acid is a C19-C24 fatty acid. In some embodiments, each fatty acid is a C19-C25 fatty acid. In some embodiments, each fatty acid is a C19-C26 fatty acid. In some embodiments, each fatty acid is a C20-C21 fatty acid. In some embodiments, each fatty acid is a C20-C22 fatty acid. In some embodiments, each fatty acid is a C20-C23 fatty acid. In some embodiments, each fatty acid is a C20-C24 fatty acid. In some embodiments, each fatty acid is a C20-C25 fatty acid. In some embodiments, each fatty acid is a C20-C26 fatty acid. In some embodiments, each fatty acid is a C21-C22 fatty acid. In some embodiments, each fatty acid is a C21-C23 fatty acid. In some embodiments, each fatty acid is a C21-C24 fatty acid. In some embodiments, each fatty acid is a C21-C25 fatty acid. In some embodiments, each fatty acid is a C21-C26 fatty acid. In some embodiments, each fatty acid is a C22-C23 fatty acid. In some embodiments, each fatty acid is a C22-C24 fatty acid. In some embodiments, each fatty acid is a C22-C25 fatty acid. In some embodiments, each fatty acid is a C22-C26 fatty acid. In some embodiments, each fatty acid is a C23-C24 fatty acid. In some embodiments, each fatty acid is a C23-C25 fatty acid. In some embodiments, each fatty acid is a C23-C26 fatty acid. In some embodiments, each fatty acid is a C24-C25 fatty acid. In some embodiments, each fatty acid is a C24-C26 fatty acid. In some embodiments, each fatty acid is a C25-C26 fatty acid. In some embodiments, each fatty acid is a C2 fatty acid. In some embodiments, each fatty acid is a C3 fatty acid. In some embodiments, each fatty acid is a C4 fatty acid. In some embodiments, each fatty acid is a C5 fatty acid. In some embodiments, each fatty acid is a C6 fatty acid. In some embodiments, each fatty acid is a C7 fatty acid. In some embodiments, each fatty acid is a C8 fatty acid. In some embodiments, each fatty acid is a C9 fatty acid. In some embodiments, each fatty acid is a C10 fatty acid. In some embodiments, each fatty acid is a C11 fatty acid. In some embodiments, each fatty acid is a C12 fatty acid. In some embodiments, each fatty acid is a C13 fatty acid. In some embodiments, each fatty acid is a C14 fatty acid. In some embodiments, each fatty acid is a C15 fatty acid. In some embodiments, each fatty acid is a C16 fatty acid. In some embodiments, each fatty acid is a C17 fatty acid. In some embodiments, each fatty acid is a C18 fatty acid. In some embodiments, each fatty acid is a C19 fatty acid. In some embodiments, each fatty acid is a C20 fatty acid. In some embodiments, each fatty acid is a C21 fatty acid. In some embodiments, each fatty acid is a C22 fatty acid. In some embodiments, each fatty acid is a C23 fatty acid. In some embodiments, each fatty acid is a C24 fatty acid. In some embodiments, each fatty acid is a C25 fatty acid. In some embodiments, each fatty acid is a C26 fatty acid.

In some embodiments, each fatty acid is independently selected from docosahexaenoic acid or eicosapentaenoic acid.

In some embodiments, each fatty acid is independently selected from docosahexaenoic acid, eicosapentaenoic acid, oleic acid, stearic acid, (9Z,12Z)-octadeca-9,12-dienoic acid, (Z)-docos-13-enoic acid, docosanoic acid, (E)-octadec-9-enoic acid, icosanoic acid, (9Z,12Z,15Z)-octadeca-9,12,15-trienoic acid, or palmitic acid.

In some embodiments, each fatty acid is independently selected from butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, and hexacosanoic acid.

In some embodiments, each fatty acid is independently selected from crotonic acid, myristoleic acid, palmitoleic acid, sapienic acid, oleic acid, elaidic acid, vaccenic acid, gadoleic acid, eicosenoic acid, erucic acid, and nervonic acid.

In some embodiments, each fatty acid is independently selected from linoleic acid, eicosadienoic acid, and docosadienoic acid.

In some embodiments, each fatty acid is independently selected from linolenic acid, pinolenic acid, eleostearic acid, mead acid, dihomo-γ-linolenic acid, and eicosatrienoic acid.

In some embodiments, each fatty acid is independently selected from stearidonic acid, arachidonic acid, eicosatetraenoic acid, and adrenic acid.

In some embodiments, the phosphorylated gemcitabine derivative prodrug of Formula (I) described herein has a structure provided in Table 1.

TABLE 1

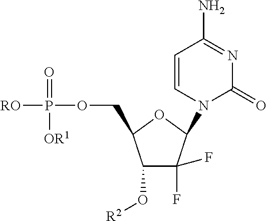

| Example | R | R¹ | R² |
|---|---|---|---|
| 1-1 | 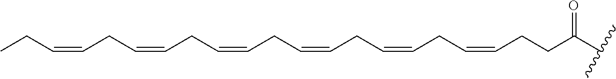 [SMILES: *C(=O)CC/C=C\C/C=C\C/C=C\C/C=C\C/C=C\C/C=C\CC] | H | H |
| 1-2 | 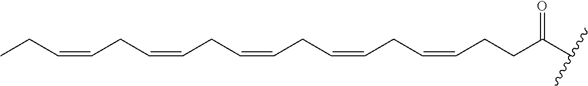 [SMILES: *C(=O)CC/C=C\C/C=C\C/C=C\C/C=C\C/C=C\CC] | H | H |
| 1-3 | 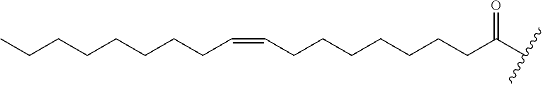 [SMILES: *C(=O)CCCCCCC/C=C\CCCCCCCC] | H | H |
| 1-4 | 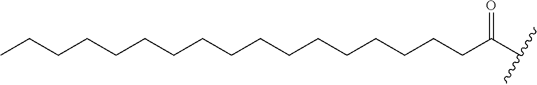 [SMILES: *C(=O)CCCCCCCCCCCCCCCCC] | H | H |
| 1-5 | 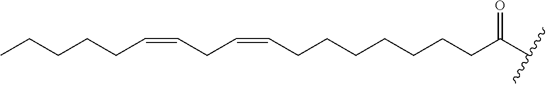 [SMILES: *C(=O)CCCCCCC/C=C\C/C=C\CCCCC] | H | H |
| 1-6 | 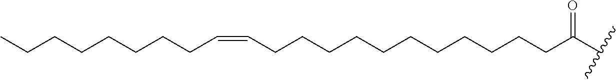 [SMILES: *C(=O)CCCCCCCCCCC/C=C\CCCCCCCC] | H | H |
| 1-7 | 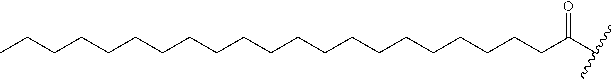 [SMILES: *C(=O)CCCCCCCCCCCCCCCCCCCCC] | H | H |
| 1-8 | 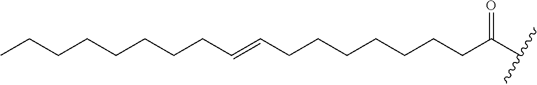 [SMILES: *C(=O)CCCCCCC/C=C/CCCCCCCC] | H | H |
| 1-9 | 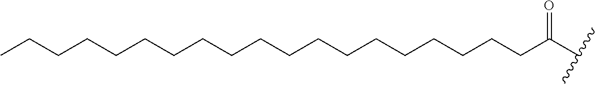 [SMILES: *C(=O)CCCCCCCCCCCCCCCCCCC] | H | H |
| 1-10 | 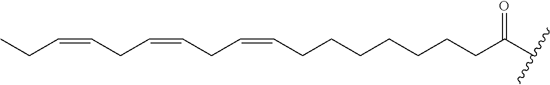 [SMILES: *C(=O)CCCCCCC/C=C\C/C=C\C/C=C\CC] | H | H |
| 1-11 | 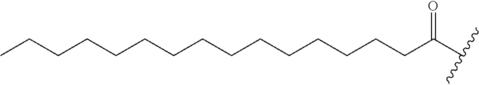 [SMILES: *C(=O)CCCCCCCCCCCCCCC] | H | H |
| 1-12 | 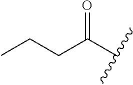 [SMILES: *C(=O)CCC] | H | H |
| 1-13 | 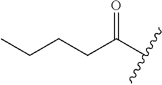 [SMILES: *C(=O)CCCC] | H | H |
| 1-14 | 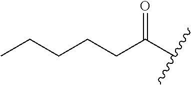 [SMILES: *C(=O)CCCCC] | H | H |
| 1-15 | 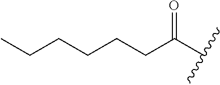 [SMILES: *C(=O)CCCCCC] | H | H |
| 1-16 | 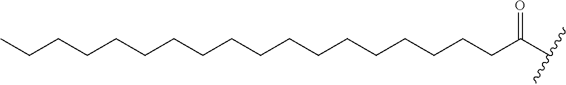 [SMILES: *C(=O)CCCCCCCCCCCCCCCCCC] | H | H |
| 1-17 | 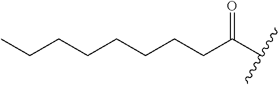 [SMILES: *C(=O)CCCCCCCC] | H | H |
| 1-18 | 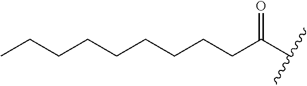 [SMILES: *C(=O)CCCCCCCCC] | H | H |
| 1-19 | 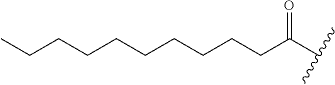 [SMILES: *C(=O)CCCCCCCCCC] | H | H |
| 1-20 | 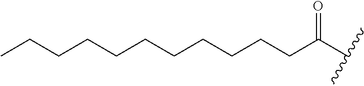 [SMILES: *C(=O)CCCCCCCCCCC] | H | H |
| 1-21 | 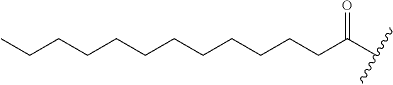 [SMILES: *C(=O)CCCCCCCCCCCC] | H | H |
| 1-22 | 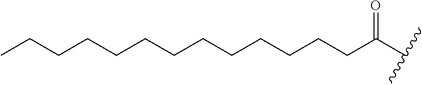 [SMILES: *C(=O)CCCCCCCCCCCCC] | H | H |
| 1-23 | 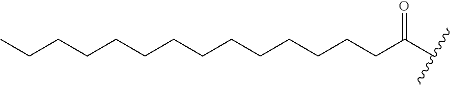 [SMILES: *C(=O)CCCCCCCCCCCCCC] | H | H |
| 1-24 | 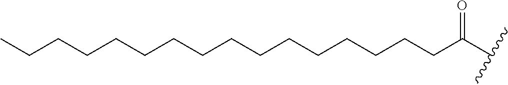 [SMILES: *C(=O)CCCCCCCCCCCCCCCC] | H | H |
| 1-25 | 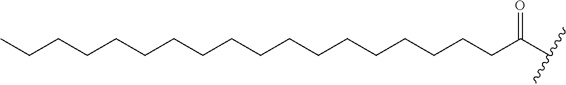 [SMILES: *C(=O)CCCCCCCCCCCCCCCCCC] | H | H |
| 1-26 | 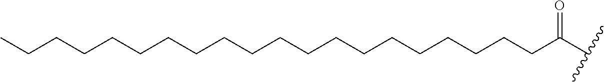 [SMILES: *C(=O)CCCCCCCCCCCCCCCCCCCC] | H | H |
| 1-27 | 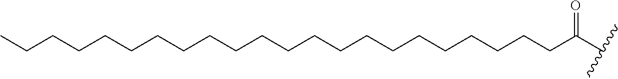 [SMILES: *C(=O)CCCCCCCCCCCCCCCCCCCCCC] | H | H |
| 1-28 | 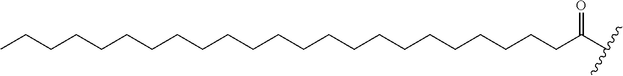 [SMILES: *C(=O)CCCCCCCCCCCCCCCCCCCCCCC] | H | H |
| 1-29 | 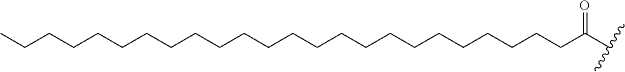 [SMILES: *C(=O)CCCCCCCCCCCCCCCCCCCCCCCC] | H | H |
| 1-30 | 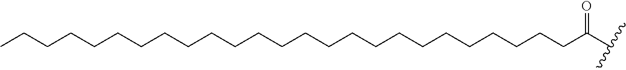 [SMILES: *C(=O)CCCCCCCCCCCCCCCCCCCCCCCCC] | H | H |
| 1-31 | 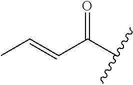 [SMILES: *C(=O)/C=C/C] | H | H |
| 1-32 | 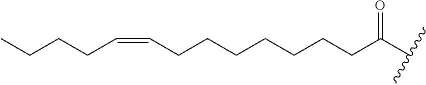 [SMILES: *C(=O)CCCCCCC/C=C\CCCC] | H | H |
| 1-33 | 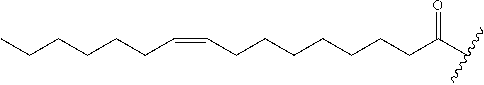 [SMILES: *C(=O)CCCCCCC/C=C\CCCCCC] | H | H |
| 1-34 | 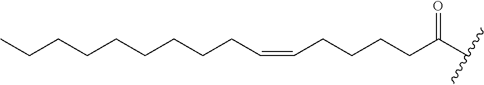 [SMILES: *C(=O)CCCC/C=C\CCCCCCCCC] | H | H |
| 1-35 | 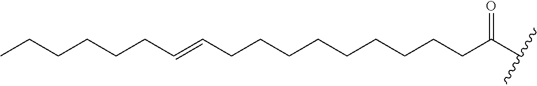 [SMILES: *C(=O)CCCCCCCCC/C=C/CCCCCC] | H | H |
| 1-36 | 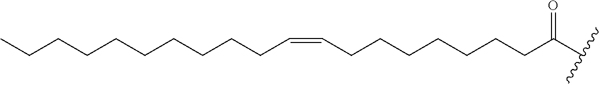 [SMILES: *C(=O)CCCCCCC/C=C\CCCCCCCCCC] | H | H |
| 1-37 | 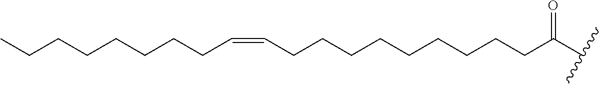 [SMILES: *C(=O)CCCCCCCCC/C=C\CCCCCCCC] | H | H |
| 1-38 | 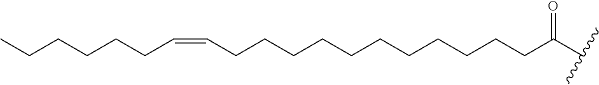 [SMILES: *C(=O)CCCCCCCCCCC/C=C\CCCCCC] | H | H |
| 1-39 | 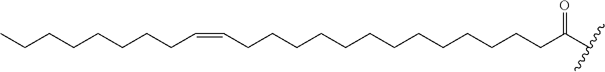 [SMILES: *C(=O)CCCCCCCCCCCCC/C=C\CCCCCCCC] | H | H |
| 1-40 | 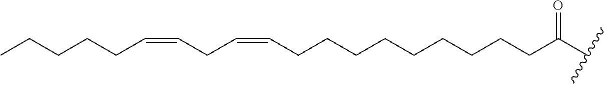 [SMILES: *C(=O)CCCCCCCCC/C=C\C/C=C\CCCCC] | H | H |
| 1-41 | 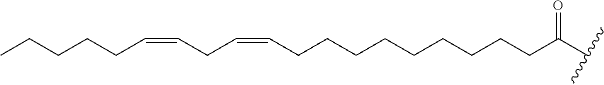 [SMILES: *C(=O)CCCCCCCCC/C=C\C/C=C\CCCCC] | H | H |
| 1-42 | 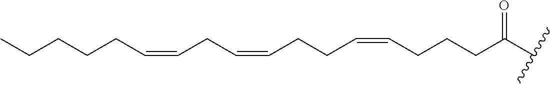 [SMILES: *C(=O)CCC/C=C\CC/C=C\C/C=C\CCCCC] | H | H |
| 1-43 | 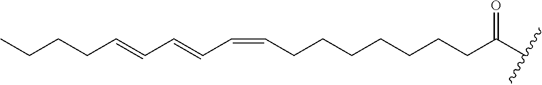 [SMILES: *C(=O)CCCCCCC\C=C/C=C/C=C/CCCC] | H | H |
| 1-44 | 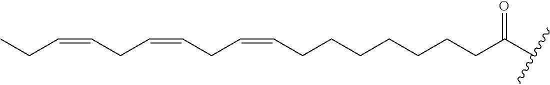 [SMILES: *C(=O)CCCCCCC/C=C\C/C=C\C/C=C\CC] | H | H |
| 1-45 | 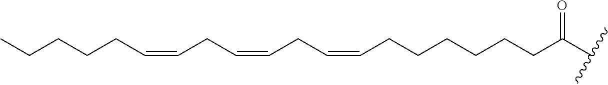 [SMILES: *C(=O)CCCCCC/C=C\C/C=C\C/C=C\CCCCC] | H | H |
| 1-46 | 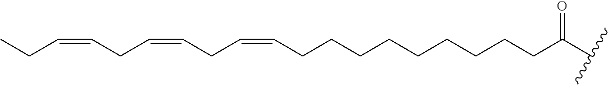 [SMILES: *C(=O)CCCCCCCCC/C=C\C/C=C\C/C=C\CC] | H | H |
| 1-47 | 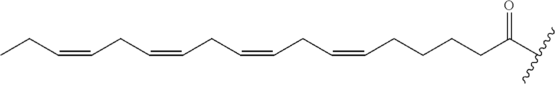 [SMILES: *C(=O)CCCC/C=C\C/C=C\C/C=C\C/C=C\CC] | H | H |
| 1-48 | 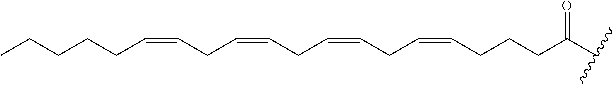 [SMILES: *C(=O)CCC/C=C\C/C=C\C/C=C\C/C=C\CCCCC] | H | H |
| 1-49 | 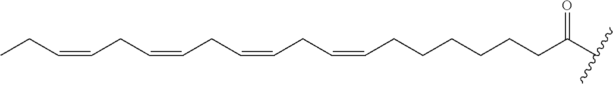 [SMILES: *C(=O)CCCCCC/C=C\C/C=C\C/C=C\C/C=C\CC] | H | H |
| 1-50 | 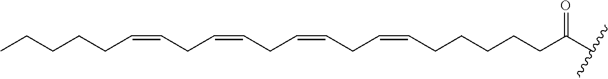 [SMILES: *C(=O)CCCCC/C=C\C/C=C\C/C=C\C/C=C\CCCCC] | H | H |
| 1-51 | 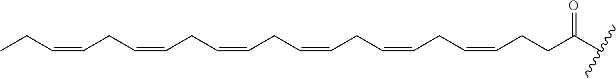 [SMILES: *C(=O)CC/C=C\C/C=C\C/C=C\C/C=C\C/C=C\C/C=C\CC] | R¹ = R | H |
| 1-52 | 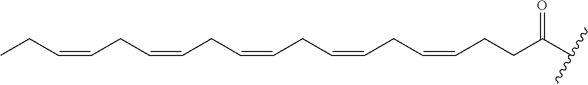 [SMILES: *C(=O)CC/C=C\C/C=C\C/C=C\C/C=C\C/C=C\CC] | R¹ = R | H |
| 1-53 | 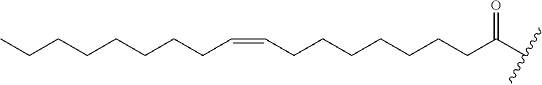 [SMILES: *C(=O)CCCCCCC/C=C\CCCCCCCC] | R¹ = R | H |
| 1-54 | 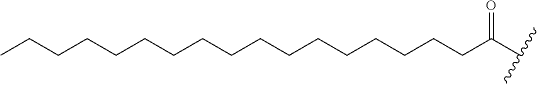 [SMILES: *C(=O)CCCCCCCCCCCCCCCCC] | R¹ = R | H |
| 1-55 | 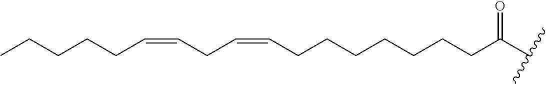 [SMILES: *C(=O)CCCCCCC/C=C\C/C=C\CCCCC] | R¹ = R | H |
| 1-56 | 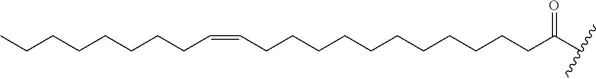 [SMILES: *C(=O)CCCCCCCCCCC/C=C\CCCCCCCC] | R¹ = R | H |
| 1-57 | 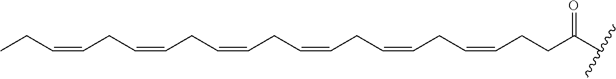 [SMILES: *C(=O)CC/C=C\C/C=C\C/C=C\C/C=C\C/C=C\C/C=C\CC] | H | R² = R |
| 1-58 | 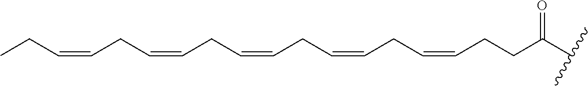 [SMILES: *C(=O)CC/C=C\C/C=C\C/C=C\C/C=C\C/C=C\CC] | H | R² = R |
| 1-59 | 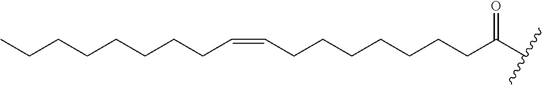 [SMILES: *C(=O)CCCCCCC/C=C\CCCCCCCC] | H | R² = R |
| 1-60 | 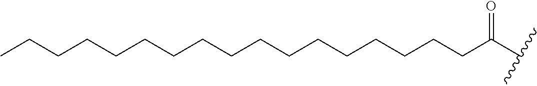 [SMILES: *C(=O)CCCCCCCCCCCCCCCCC] | H | R² = R |
| 1-61 | 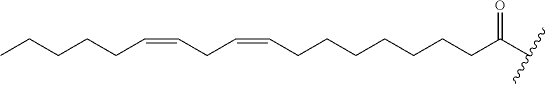 [SMILES: *C(=O)CCCCCCC/C=C\C/C=C\CCCCC] | H | R² = R |
| 1-62 | 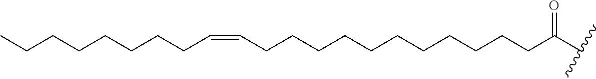 [SMILES: *C(=O)CCCCCCCCCCC/C=C\CCCCCCCC] | H | R² = R |

In another aspect, provided herein is a compound, or pharmaceutically acceptable salt thereof, having a structure provided in Formula (II),

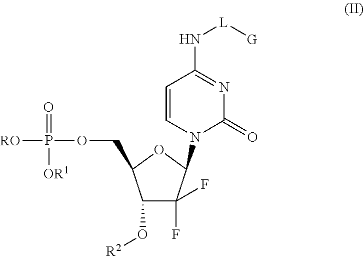

wherein,

R is selected from fatty acid, glycerolipid, glycerophospholipids, sphingolipids, sterol-lipids, prenol lipids, saccharolipids and polyketides;

R¹ is selected from hydrogen, fatty acid, glycerolipid, glycerophospholipids, sphingolipids, sterol-lipids, prenol lipids, saccharolipids and polyketides;

R² is selected from hydrogen, fatty acid, glycerolipid, glycerophospholipids, sphingolipids, sterol-lipids, prenol lipids, saccharolipids and polyketides;

L is a linker selected from an alkylene amide group, an alkylene ester group, an alkylene carbamate group, a disulfide group, a phosphodiester group, and a phosphoramidate group; and

G is a cytotoxic chemotherapy agent.

In some embodiments, the cytotoxic chemotherapy agent is selected from methotrexate, doxorubicin, vincristine, procarbazine, prednisolone, bleomycin, vinblastine, dacarbazine, bleomycin, etoposide, curcumin, SN38, rapamycin, geldanamycin, tanespimycin, or IDN5404.

In some embodiments, the cytotoxic chemotherapy agent is selected from abiraterone, afatinib, axitinib, azacitidine, bortezomib, cabazitaxel, cabozantinib, capecitabine, carfilzomib, ceritinib, crizotinib, cyclophosphamide, cytarabine, dabrafenib, dactinomycin, dasatinib, daunorubicin, decarbazine, decitabine, docetaxel, doxorubicin, epirubicin, erlotinib, etoposide, everrolimus, floxuridine, gefitinib, ibrutinib, idarubicin, idelalisib, lapatinib, lenvatinib, leucovorin, methotrexate, mitomycin, olaparib, palbociclib, pazopanib, ponatinib, pralatrexate, prednisone, regorafenib, ruxolitinib, sorafenib, streptozocin, sunitinib, thalidomide, topotecan, vemurafenib, vincristine, vinorelbine, and zoledronic acid.

In some embodiments, L is an alkylene carbamate group.

In some embodiments, R¹ is hydrogen. In some embodiments, R² is hydrogen. In some embodiments, R¹ and R² are hydrogen.

In some embodiments, R¹ is not hydrogen. In some embodiments, R¹ is not hydrogen, and R and R¹ are selected independently. In some embodiments, R¹ is not hydrogen, and R and R¹ are the same. In some embodiments, R¹ is not hydrogen and R² is hydrogen.

In some embodiments, R² is not hydrogen. In some embodiments, R² is not hydrogen, and R and R² are selected independently. In some embodiments, R² is not hydrogen, and R and R² are the same. In some embodiments, R¹ is hydrogen and R² is not hydrogen.

In some embodiments, R and R¹ are independently selected from a fatty acid. In some embodiments, R is a fatty acid, and R¹ is hydrogen. In some embodiments, R and R² are independently selected from a fatty acid. In some embodiments, R is a fatty acid, and R² is hydrogen.

In some embodiments, R¹ and R² are both hydrogen.

In some embodiments, each fatty acid is independently selected from a saturated, monounsaturated, or polyunsaturated fatty acid.

In some embodiments, each fatty acid is a C2-C26 fatty acid. In some embodiments, each fatty acid is a C2-C3 fatty acid. In some embodiments, each fatty acid is a C2-C4 fatty acid. In some embodiments, each fatty acid is a C2-C5 fatty acid. In some embodiments, each fatty acid is a C2-C6 fatty acid. In some embodiments, each fatty acid is a C2-C7 fatty acid. In some embodiments, each fatty acid is a C2-C8 fatty acid. In some embodiments, each fatty acid is a C2-C9 fatty acid. In some embodiments, each fatty acid is a C2-C10 fatty acid. In some embodiments, each fatty acid is a C2-C11 fatty acid. In some embodiments, each fatty acid is a C2-C12 fatty acid. In some embodiments, each fatty acid is a C2-C13 fatty acid. In some embodiments, each fatty acid is a C2-C14 fatty acid. In some embodiments, each fatty acid is a C2-C15 fatty acid. In some embodiments, each fatty acid is a C2-C16 fatty acid. In some embodiments, each fatty acid is a C2-C17 fatty acid. In some embodiments, each fatty acid is a C2-C18 fatty acid. In some embodiments, each fatty acid is a C2-C19 fatty acid. In some embodiments, each fatty acid is a C2-C20 fatty acid. In some embodiments, each fatty acid is a C2-C21 fatty acid. In some embodiments, each fatty acid is a C2-C22 fatty acid. In some embodiments, each fatty acid is a C2-C23 fatty acid. In some embodiments, each fatty acid is a C2-C24 fatty acid. In some embodiments, each fatty acid is a C2-C25 fatty acid. In some embodiments, each fatty acid is a C2-C26 fatty acid. In some embodiments, each fatty acid is a C3-C4 fatty acid. In some embodiments, each fatty acid is a C3-C5 fatty acid. In some embodiments, each fatty acid is a C3-C6 fatty acid. In some embodiments, each fatty acid is a C3-C7 fatty acid. In some embodiments, each fatty acid is a C3-C8 fatty acid. In some embodiments, each fatty acid is a C3-C9 fatty acid. In some embodiments, each fatty acid is a C3-C10 fatty acid. In some embodiments, each fatty acid is a C3-C11 fatty acid. In some embodiments, each fatty acid is a C3-C12 fatty acid. In some embodiments, each fatty acid is a C3-C13 fatty acid. In some embodiments, each fatty acid is a C3-C14 fatty acid. In some embodiments, each fatty acid is a C3-C15 fatty acid. In some embodiments, each fatty acid is a C3-C16 fatty acid. In some embodiments, each fatty acid is a C3-C17 fatty acid. In some embodiments, each fatty acid is a C3-C18 fatty acid. In some embodiments, each fatty acid is a C3-C19 fatty acid. In some embodiments, each fatty acid is a C3-C20 fatty acid. In some embodiments, each fatty acid is a C3-C21 fatty acid. In some embodiments, each fatty acid is a C3-C22 fatty acid. In some embodiments, each fatty acid is a C3-C23 fatty acid. In some embodiments, each fatty acid is a C3-C24 fatty acid. In some embodiments, each fatty acid is a C3-C25 fatty acid. In some embodiments, each fatty acid is a C3-C26 fatty acid. In some embodiments, each fatty acid is a C4-C5 fatty acid. In some embodiments, each fatty acid is a C4-C6 fatty acid. In some embodiments, each fatty acid is a C4-C7 fatty acid. In some embodiments, each fatty acid is a C4-C8 fatty acid. In some embodiments, each fatty acid is a C4-C9 fatty acid. In some embodiments, each fatty acid is a C4-C10 fatty acid. In some embodiments, each fatty acid is a C4-C11 fatty acid. In some embodiments, each fatty acid is a C4-C12 fatty acid. In some embodiments, each fatty acid is a C4-C13 fatty acid. In some embodiments, each fatty acid is a C4-C14 fatty acid. In some embodiments, each fatty acid is a C4-C15 fatty acid. In some embodiments, each fatty acid is a C4-C16 fatty acid. In some embodiments, each fatty acid is a C4-C17 fatty acid. In some embodiments, each fatty acid is a C4-C18 fatty acid. In some embodiments, each fatty acid is a C4-C19 fatty acid. In some embodiments, each fatty acid is a C4-C20 fatty acid. In some embodiments, each fatty acid is a C4-C21 fatty acid. In some embodiments, each fatty acid is a C4-C22 fatty acid. In some embodiments, each fatty acid is a C4-C23 fatty acid. In some embodiments, each fatty acid is a C4-C24 fatty acid. In some embodiments, each fatty acid is a C4-C25 fatty acid. In some embodiments, each fatty acid is a C4-C26 fatty acid. In some embodiments, each fatty acid is a C5-C6 fatty acid. In some embodiments, each fatty acid is a C5-C7 fatty acid. In some embodiments, each fatty acid is a C5-C8 fatty acid. In some embodiments, each fatty acid is a C5-C9 fatty acid. In some embodiments, each fatty acid is a C5-C10 fatty acid. In some embodiments, each fatty acid is a C5-C11 fatty acid. In some embodiments, each fatty acid is a C5-C12 fatty acid. In some embodiments, each fatty acid is a C5-C13 fatty acid. In some embodiments, each fatty acid is a C5-C14 fatty acid. In some embodiments, each fatty acid is a C5-C15 fatty acid. In some embodiments, each fatty acid is a C5-C16 fatty acid. In some embodiments, each fatty acid is a C5-C17 fatty acid. In some embodiments, each fatty acid is a C5-C18 fatty acid. In some embodiments, each fatty acid is a C5-C19 fatty acid. In some embodiments, each fatty acid is a C5-C20 fatty acid. In some embodiments, each fatty acid is a C5-C21 fatty acid. In some embodiments, each fatty acid is a C5-C22 fatty acid. In some embodiments, each fatty acid is a C5-C23 fatty acid. In some embodiments, each fatty acid is a C5-C24 fatty acid. In some embodiments, each fatty acid is a C5-C25 fatty acid. In some embodiments, each fatty acid is a C5-C26 fatty acid. In some embodiments, each fatty acid is a C6-C7 fatty acid. In some embodiments, each fatty acid is a C6-C8 fatty acid. In some embodiments, each fatty acid is a C6-C9 fatty acid. In some embodiments, each fatty acid is a C6-C10 fatty acid. In some embodiments, each fatty acid is a C6-C11 fatty acid. In some embodiments, each fatty acid is a C6-C12 fatty acid. In some embodiments, each fatty acid is a C6-C13 fatty acid. In some embodiments, each fatty acid is a C6-C14 fatty acid. In some embodiments, each fatty acid is a C6-C15 fatty acid. In some embodiments, each fatty acid is a C6-C16 fatty acid. In some embodiments, each fatty acid is a C6-C17 fatty acid. In some embodiments, each fatty acid is a C6-C18 fatty acid. In some embodiments, each fatty acid is a C6-C19 fatty acid. In some embodiments, each fatty acid is a C6-C20 fatty acid. In some embodiments, each fatty acid is a C6-C21 fatty acid. In some embodiments, each fatty acid is a C6-C22 fatty acid. In some embodiments, each fatty acid is a C6-C23 fatty acid. In some embodiments, each fatty acid is a C6-C24 fatty acid. In some embodiments, each fatty acid is a C6-C25 fatty acid. In some embodiments, each fatty acid is a C6-C26 fatty acid. In some embodiments, each fatty acid is a C7-C8 fatty acid. In some embodiments, each fatty acid is a C7-C9 fatty acid. In some embodiments, each fatty acid is a C7-C10 fatty acid. In some embodiments, each fatty acid is a C7-C11 fatty acid. In some embodiments, each fatty acid is a C7-C12 fatty acid. In some embodiments, each fatty acid is a C7-C13 fatty acid. In some embodiments, each fatty acid is a C7-C14 fatty acid. In some embodiments, each fatty acid is a C7-C15 fatty acid. In some embodiments, each fatty acid is a C7-C16 fatty acid. In some embodiments, each fatty acid is a C7-C17 fatty acid. In some embodiments, each fatty acid is a C7-C18 fatty acid. In some embodiments, each fatty acid is a C7-C19 fatty acid. In some embodiments, each fatty acid is a C7-C20 fatty acid. In some embodiments, each fatty acid is a C7-C21 fatty acid. In some embodiments, each fatty acid is a C7-C22 fatty acid. In some embodiments, each fatty acid is a C7-C23 fatty acid. In some embodiments, each fatty acid is a C7-C24 fatty acid. In some embodiments, each fatty acid is a C7-C25 fatty acid. In some embodiments, each fatty acid is a C7-C26 fatty acid. In some embodiments, each fatty acid is a C8-C9 fatty acid. In some embodiments, each fatty acid is a C8-C10 fatty acid. In some embodiments, each fatty acid is a C8-C11 fatty acid. In some embodiments, each fatty acid is a C8-C12 fatty acid. In some embodiments, each fatty acid is a C8-C13 fatty acid. In some embodiments, each fatty acid is a C8-C14 fatty acid. In some embodiments, each fatty acid is a C8-C15 fatty acid. In some embodiments, each fatty acid is a C8-C16 fatty acid. In some embodiments, each fatty acid is a C8-C17 fatty acid. In some embodiments, each fatty acid is a C8-C18 fatty acid. In some embodiments, each fatty acid is a C8-C19 fatty acid. In some embodiments, each fatty acid is a C8-C20 fatty acid. In some embodiments, each fatty acid is a C8-C21 fatty acid. In some embodiments, each fatty acid is a C8-C22 fatty acid. In some embodiments, each fatty acid is a C8-C23 fatty acid. In some embodiments, each fatty acid is a C8-C24 fatty acid. In some embodiments, each fatty acid is a C8-C25 fatty acid. In some embodiments, each fatty acid is a C8-C26 fatty acid. In some embodiments, each fatty acid is a C9-C10 fatty acid. In some embodiments, each fatty acid is a C9-C11 fatty acid. In some embodiments, each fatty acid is a C9-C12 fatty acid. In some embodiments, each fatty acid is a C9-C13 fatty acid. In some embodiments, each fatty acid is a C9-C14 fatty acid. In some embodiments, each fatty acid is a C9-C15 fatty acid. In some embodiments, each fatty acid is a C9-C16 fatty acid. In some embodiments, each fatty acid is a C9-C17 fatty acid. In some embodiments, each fatty acid is a C9-C18 fatty acid. In some embodiments, each fatty acid is a C9-C19 fatty acid. In some embodiments, each fatty acid is a C9-C20 fatty acid. In some embodiments, each fatty acid is a C9-C21 fatty acid. In some embodiments, each fatty acid is a C9-C22 fatty acid. In some embodiments, each fatty acid is a C9-C23 fatty acid. In some embodiments, each fatty acid is a C9-C24 fatty acid. In some embodiments, each fatty acid is a C9-C25 fatty acid. In some embodiments, each fatty acid is a C9-C26 fatty acid. In some embodiments, each fatty acid is a C10-C11 fatty acid. In some embodiments, each fatty acid is a C10-C12 fatty acid. In some embodiments, each fatty acid is a C10-C13 fatty acid. In some embodiments, each fatty acid is a C10-C14 fatty acid. In some embodiments, each fatty acid is a C10-C15 fatty acid. In some embodiments, each fatty acid is a C10-C16 fatty acid. In some embodiments, each fatty acid is a C10-C17 fatty acid. In some embodiments, each fatty acid is a C10-C18 fatty acid. In some embodiments, each fatty acid is a C10-C19 fatty acid. In some embodiments, each fatty acid is a C10-C20 fatty acid. In some embodiments, each fatty acid is a C10-C21 fatty acid. In some embodiments, each fatty acid is a C10-C22 fatty acid. In some embodiments, each fatty acid is a C10-C23 fatty acid. In some embodiments, each fatty acid is a C10-C24 fatty acid. In some embodiments, each fatty acid is a C10-C25 fatty acid. In some embodiments, each fatty acid is a C10-C26 fatty acid. In some embodiments, each fatty acid is a C11-C12 fatty acid. In some embodiments, each fatty acid is a C11-C13 fatty acid. In some embodiments, each fatty acid is a C11-C14 fatty acid. In some embodiments, each fatty acid is a C11-C15 fatty acid. In some embodiments, each fatty acid is a C11-C16 fatty acid. In some embodiments, each fatty acid is a C11-C17 fatty acid. In some embodiments, each fatty acid is a C11-C18 fatty acid. In some embodiments, each fatty acid is a C11-C19 fatty acid. In some embodiments, each fatty acid is a C11-C20 fatty acid. In some embodiments, each fatty acid is a C11-C21 fatty acid. In some embodiments, each fatty acid is a C11-C22 fatty acid. In some embodiments, each fatty acid is a C11-C23 fatty acid. In some embodiments, each fatty acid is a C11-C24 fatty acid. In some embodiments, each fatty acid is a C11-C25 fatty acid. In some embodiments, each fatty acid is a C11-C26 fatty acid. In some embodiments, each fatty acid is a C12-C13 fatty acid. In some embodiments, each fatty acid is a C12-C14 fatty acid. In some embodiments, each fatty acid is a C12-C15 fatty acid. In some embodiments, each fatty acid is a C12-C16 fatty acid. In some embodiments, each fatty acid is a C12-C17 fatty acid. In some embodiments, each fatty acid is a C12-C18 fatty acid. In some embodiments, each fatty acid is a C12-C19 fatty acid. In some embodiments, each fatty acid is a C12-C20 fatty acid. In some embodiments, each fatty acid is a C12-C21 fatty acid. In some embodiments, each fatty acid is a C12-C22 fatty acid. In some embodiments, each fatty acid is a C12-C23 fatty acid. In some embodiments, each fatty acid is a C12-C24 fatty acid. In some embodiments, each fatty acid is a C12-C25 fatty acid. In some embodiments, each fatty acid is a C12-C26 fatty acid. In some embodiments, each fatty acid is a C13-C14 fatty acid. In some embodiments, each fatty acid is a C13-C15 fatty acid. In some embodiments, each fatty acid is a C13-C16 fatty acid. In some embodiments, each fatty acid is a C13-C17 fatty acid. In some embodiments, each fatty acid is a C13-C18 fatty acid. In some embodiments, each fatty acid is a C13-C19 fatty acid. In some embodiments, each fatty acid is a C13-C20 fatty acid. In some embodiments, each fatty acid is a C13-C21 fatty acid. In some embodiments, each fatty acid is a C13-C22 fatty acid. In some embodiments, each fatty acid is a C13-C23 fatty acid. In some embodiments, each fatty acid is a C13-C24 fatty acid. In some embodiments, each fatty acid is a C13-C25 fatty acid. In some embodiments, each fatty acid is a C13-C26 fatty acid. In some embodiments, each fatty acid is a C14-C15 fatty acid. In some embodiments, each fatty acid is a C14-C16 fatty acid. In some embodiments, each fatty acid is a C14-C17 fatty acid. In some embodiments, each fatty acid is a C14-C18 fatty acid. In some embodiments, each fatty acid is a C14-C19 fatty acid. In some embodiments, each fatty acid is a C14-C20 fatty acid. In some embodiments, each fatty acid is a C14-C21 fatty acid. In some embodiments, each fatty acid is a C14-C22 fatty acid. In some embodiments, each fatty acid is a C14-C23 fatty acid. In some embodiments, each fatty acid is a C14-C24 fatty acid. In some embodiments, each fatty acid is a C14-C25 fatty acid. In some embodiments, each fatty acid is a C14-C26 fatty acid. In some embodiments, each fatty acid is a C15-C16 fatty acid. In some embodiments, each fatty acid is a C15-C17 fatty acid. In some embodiments, each fatty acid is a C15-C18 fatty acid. In some embodiments, each fatty acid is a C15-C19 fatty acid. In some embodiments, each fatty acid is a C15-C20 fatty acid. In some embodiments, each fatty acid is a C15-C21 fatty acid. In some embodiments, each fatty acid is a C15-C22 fatty acid. In some embodiments, each fatty acid is a C15-C23 fatty acid. In some embodiments, each fatty acid is a C15-C24 fatty acid. In some embodiments, each fatty acid is a C15-C25 fatty acid. In some embodiments, each fatty acid is a C15-C26 fatty acid. In some embodiments, each fatty acid is a C16-C17 fatty acid. In some embodiments, each fatty acid is a C16-C18 fatty acid. In some embodiments, each fatty acid is a C16-C19 fatty acid. In some embodiments, each fatty acid is a C16-C20 fatty acid. In some embodiments, each fatty acid is a C16-C21 fatty acid. In some embodiments, each fatty acid is a C16-C22 fatty acid. In some embodiments, each fatty acid is a C16-C23 fatty acid. In some embodiments, each fatty acid is a C16-C24 fatty acid. In some embodiments, each fatty acid is a C16-C25 fatty acid. In some embodiments, each fatty acid is a C16-C26 fatty acid. In some embodiments, each fatty acid is a C17-C18 fatty acid. In some embodiments, each fatty acid is a C17-C19 fatty acid. In some embodiments, each fatty acid is a C17-C20 fatty acid. In some embodiments, each fatty acid is a C17-C21 fatty acid. In some embodiments, each fatty acid is a C17-C22 fatty acid. In some embodiments, each fatty acid is a C17-C23 fatty acid. In some embodiments, each fatty acid is a C17-C24 fatty acid. In some embodiments, each fatty acid is a C17-C25 fatty acid. In some embodiments, each fatty acid is a C17-C26 fatty acid. In some embodiments, each fatty acid is a C18-C19 fatty acid. In some embodiments, each fatty acid is a C18-C20 fatty acid. In some embodiments, each fatty acid is a C18-C21 fatty acid. In some embodiments, each fatty acid is a C18-C22 fatty acid. In some embodiments, each fatty acid is a C18-C23 fatty acid. In some embodiments, each fatty acid is a C18-C24 fatty acid. In some embodiments, each fatty acid is a C18-C25 fatty acid. In some embodiments, each fatty acid is a C18-C26 fatty acid. In some embodiments, each fatty acid is a C19-C20 fatty acid. In some embodiments, each fatty acid is a C19-C21 fatty acid. In some embodiments, each fatty acid is a C19-C22 fatty acid. In some embodiments, each fatty acid is a C19-C23 fatty acid. In some embodiments, each fatty acid is a C19-C24 fatty acid. In some embodiments, each fatty acid is a C19-C25 fatty acid. In some embodiments, each fatty acid is a C19-C26 fatty acid. In some embodiments, each fatty acid is a C20-C21 fatty acid. In some embodiments, each fatty acid is a C20-C22 fatty acid. In some embodiments, each fatty acid is a C20-C23 fatty acid. In some embodiments, each fatty acid is a C20-C24 fatty acid. In some embodiments, each fatty acid is a C20-C25 fatty acid. In some embodiments, each fatty acid is a C20-C26 fatty acid. In some embodiments, each fatty acid is a C21-C22 fatty acid. In some embodiments, each fatty acid is a C21-C23 fatty acid. In some embodiments, each fatty acid is a C21-C24 fatty acid. In some embodiments, each fatty acid is a C21-C25 fatty acid. In some embodiments, each fatty acid is a C21-C26 fatty acid. In some embodiments, each fatty acid is a C22-C23 fatty acid. In some embodiments, each fatty acid is a C22-C24 fatty acid. In some embodiments, each fatty acid is a C22-C25 fatty acid. In some embodiments, each fatty acid is a C22-C26 fatty acid. In some embodiments, each fatty acid is a C23-C24 fatty acid. In some embodiments, each fatty acid is a C23-C25 fatty acid. In some embodiments, each fatty acid is a C23-C26 fatty acid. In some embodiments, each fatty acid is a C24-C25 fatty acid. In some embodiments, each fatty acid is a C24-C26 fatty acid. In some embodiments, each fatty acid is a C25-C26 fatty acid. In some embodiments, each fatty acid is a C2 fatty acid. In some embodiments, each fatty acid is a C3 fatty acid. In some embodiments, each fatty acid is a C4 fatty acid. In some embodiments, each fatty acid is a C5 fatty acid. In some embodiments, each fatty acid is a C6 fatty acid. In some embodiments, each fatty acid is a C7 fatty acid. In some embodiments, each fatty acid is a C8 fatty acid. In some embodiments, each fatty acid is a C9 fatty acid. In some embodiments, each fatty acid is a C10 fatty acid. In some embodiments, each fatty acid is a C11 fatty acid. In some embodiments, each fatty acid is a C12 fatty acid. In some embodiments, each fatty acid is a C13 fatty acid. In some embodiments, each fatty acid is a C14 fatty acid. In some embodiments, each fatty acid is a C15 fatty acid. In some embodiments, each fatty acid is a C16 fatty acid. In some embodiments, each fatty acid is a C17 fatty acid. In some embodiments, each fatty acid is a C18 fatty acid. In some embodiments, each fatty acid is a C19 fatty acid. In some embodiments, each fatty acid is a C20 fatty acid. In some embodiments, each fatty acid is a C21 fatty acid. In some embodiments, each fatty acid is a C22 fatty acid. In some embodiments, each fatty acid is a C23 fatty acid. In some embodiments, each fatty acid is a C24 fatty acid. In some embodiments, each fatty acid is a C25 fatty acid. In some embodiments, each fatty acid is a C26 fatty acid.

In some embodiments, each fatty acid is independently selected from docosahexaenoic acid or eicosapentaenoic acid.

In some embodiments, each fatty acid is independently selected from docosahexaenoic acid, eicosapentaenoic acid, oleic acid, stearic acid, (9Z,12Z)-octadeca-9,12-dienoic acid, (Z)-docos-13-enoic acid, docosanoic acid, (E)-octadec-9-enoic acid, icosanoic acid, (9Z,12Z,15Z)-octadeca-9,12,15-trienoic acid, or palmitic acid.

In some embodiments, each fatty acid is independently selected from butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, and hexacosanoic acid.

In some embodiments, each fatty acid is independently selected from crotonic acid, myristoleic acid, palmitoleic acid, sapienic acid, oleic acid, elaidic acid, vaccenic acid, gadoleic acid, eicosenoic acid, erucic acid, and nervonic acid.

In some embodiments, each fatty acid is independently selected from linoleic acid, eicosadienoic acid, and docosadienoic acid.

In some embodiments, each fatty acid is independently selected from linolenic acid, pinolenic acid, eleostearic acid, mead acid, dihomo-γ-linolenic acid, and eicosatrienoic acid.

In some embodiments, each fatty acid is independently selected from stearidonic acid, arachidonic acid, eicosatetraenoic acid, and adrenic acid.

In some embodiments, the compound of Formula (II) has the structure of Formula (IIa):

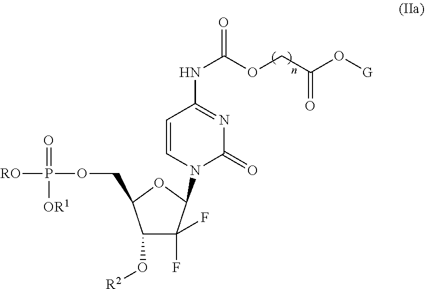

wherein n is 1 to 6.

In some embodiments, R¹ is hydrogen.

In some embodiments, R and R¹ are independently selected from a fatty acid.

In some embodiments, each fatty acid is independently selected from a saturated, monounsaturated, or polyunsaturated fatty acid.

In some embodiments, each fatty acid is independently selected from docosahexaenoic acid or eicosapentaenoic acid.

In some embodiments, each fatty acid is independently selected from docosahexaenoic acid, eicosapentaenoic acid, oleic acid, stearic acid, (9Z,12Z)-octadeca-9,12-dienoic acid, (Z)-docos-13-enoic acid, docosanoic acid, (E)-octadec-9-enoic acid, icosanoic acid, (9Z,12Z,15Z)-octadeca-9,12,15-trienoic acid, or palmitic acid.

In some embodiments, each fatty acid is independently selected from butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, and hexacosanoic acid.

In some embodiments, each fatty acid is independently selected from crotonic acid, myristoleic acid, palmitoleic acid, sapienic acid, oleic acid, elaidic acid, vaccenic acid, gadoleic acid, eicosenoic acid, erucic acid, and nervonic acid.

In some embodiments, each fatty acid is independently selected from linoleic acid, eicosadienoic acid, and docosadienoic acid.

In some embodiments, each fatty acid is independently selected from linolenic acid, pinolenic acid, eleostearic acid, mead acid, dihomo-γ-linolenic acid, and eicosatrienoic acid.

In some embodiments, each fatty acid is independently selected from stearidonic acid, arachidonic acid, eicosatetraenoic acid, and adrenic acid.

Preparation of Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, CN), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wileylnterscience, N.Y., 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Specific and analogous reactants are optionally identified through the indices of known chemicals prepared by the Chemical Abstract Service of the American Chemical Society, which are available in most public and university libraries, as well as through online databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the gemcitabine prodrug compounds described herein is P. H. Stahl & C. G. Wermuth “Handbook of Pharmaceutical Salts”, Verlag Helvetica Chimica Acta, Zurich, 2002.

Pharmaceutical Compositions

In certain embodiments, the phosphorylated gemcitabine derivative prodrug as described herein is administered as a pure chemical. In other embodiments, the phosphorylated gemcitabine derivative prodrug described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, Pa. (2005)).

Provided herein is a pharmaceutical composition comprising at least one phosphorylated gemcitabine derivative prodrug, or a stereoisomer, pharmaceutically acceptable salt, hydrate, or solvate thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising a pharmaceutically acceptable excipient and a compound of any one of Formula (I), (II), and (IIa), or a compound disclosed in Table 1, or a pharmaceutically acceptable salt thereof.

In certain embodiments, the phosphorylated gemcitabine derivative prodrug as described by any one of Formula (I), (II), and (IIa), or a compound disclosed in Table 1, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21st Ed. Mack Pub. Co., Easton, Pa. (2005)).

In some embodiments, the phosphorylated gemcitabine derivative prodrug as described by any one of Formula (I), (II), and (IIa), or a compound disclosed in Table 1, or pharmaceutically acceptable salt thereof, is formulated for administration by injection. In some instances, the injection formulation is an aqueous formulation. In some instances, the injection formulation is a non-aqueous formulation. In some instances, the injection formulation is an oil-based formulation, such as sesame oil, or the like.

In some embodiments, the phosphorylated gemcitabine derivative prodrug as described by any one of Formula (I), (II), and (IIa), or a compound disclosed in Table 1, or pharmaceutically acceptable salt thereof, is formulated as a nanoparticle. Various forms of nanoparticles are contemplated herein. In some embodiments, the nanoparticle comprises an outer layer comprising a polymer or protein such as albumin while an inner core contains the gemcitabine derivative prodrug. In some embodiments, the nanoparticle comprises a mixture of polymer or protein. For example, the mixture can comprise albumin and the gemcitabine derivative prodrug. Nanoparticles can exhibit less toxicity, higher activity, higher distributions at drug target sites (including tumors), better uptake, and better efficacy as compared to free gemcitabine or free gemcitabine derivative prodrugs. In some embodiments, the use of a lipophilic gemcitabine derivative prodrug can be advantageous in nanoparticle formulations, for example, by increasing or enhacing binding or compatibility of the prodrug with the materials in core of the nanoparticle, release of the prodrug at the site of desired activity, or transport within the body.

In some embodiments, the nanoparticle comprises a calcium phosphate lipid nanoparticle. In some embodiments, the nanoparticle comprises a lipid-based nanoparticle. In some embodiments, the phosphorylated gemcitabine derivative prodrug is incorporated in the lipid bilayer of a liposomal nanoparticle. In some embodiments, the phosphorylated gemcitabine derivative prodrug is incorporated inside the liposomal nanoparticle. In some embodiments, the nanoparticle comprises an albumin-coated nanoparticle. In some embodiments, the albumin-coated nanoparticle comprises albumin covalently bound, non-covalently bound, or both covalently and non-covalently bound to the gemcitabine derivative prodrugs described herein. Exemplary methods of covalently binding the prodrug to albumin include chemical conjugation of the drug, which includes conjugation to lysines, tyrosines, or the free SH-group on the cys34. Such conjugations can include conjugations using chemical linkers, including, for example, maleimide or an an acid sensitive hydrazone linker. In some embodiments, the phosphorylated gemcitabine derivative prodrug is incorporated in a polymeric nanoparticle. In some embodiments, the phosphorylated gemcitabine derivative prodrug is incorporated in a polymeric or lipid nanoparticle that is coated with polyethylene glycol polymer. In some embodiments, the polymeric nanoparticle comprises a core made of biodegradable polymers such as poly lactic acid, poly glycolic acid or copolymers of poly (lactic-co-glycolic acid), poly caprolactone, copolymers of poly (caprolactone-co-lactic acid), poly (caprolactone-co-glycolic acid). In some embodiments, the molecular weight of the biogdegradable polymers ranges from 500 daltons to 5000 daltons. In some embodiments, the molecular weight of the biogdegradable polymers ranges from 5000 daltons to 100000 daltons. In some embodiments, the poly ethylene glycol polymer molecular weight of polyethylene glycol is 500 to 20000 daltons.

In some embodiments, the phosphorylated gemcitabine derivative prodrug as described by any one of Formula (I), (II), and (IIa), or a compound disclosed in Table 1, or pharmaceutically acceptable salt thereof, is formulated as or comprises a hydrogel, a liposome, a polymeric nanoparticle, a silica-based nanoparticle, a dendrimer, a nanotube, a polymersome, a quantum dot, and/or an XPclad nanoparticle.

In some embodiments, the nanoparticle has a diameter between about 10 nm and about 10000 nm. In some embodiments, the nanoparticle has a diameter between about 30 nm and about 70 nm, between about 70 nm and about 120 nm, between about 120 nm and about 200 nm, between about 200 nm and about 5000 nm, or between about 500 nm and about 1000 nm. The dose of the composition comprising at least one phosphorylated gemcitabine derivative prodrug as described herein differ depending upon the subject or patient's (e.g., human) condition. Such factors can include general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Dosing and Therapeutic Regimens

In some embodiments, the pharmaceutical compositions described herein are administered for therapeutic applications. In some embodiments, the pharmaceutical composition is administered once per day, twice per day, three times per day, four times per day or more. The pharmaceutical composition is administered daily, every day, every alternate day, two days a week, three days a week, four days a week, five days a week, once a week, every other week, two weeks per month, three weeks per month, once a month, twice a month, three times per month, or more. The pharmaceutical composition is administered for at least 1 week, 2 weeks, 1 month, 2 months, 3 months, 4 months, 5 months, 6 months, 7 months, 8 months, 9 months, 10 months, 11 months, 12 months, 18 months, 2 years, 3 years, or more.

In the case wherein the patient's status does not improve, upon the physician's discretion the administration of the composition is given continuously; alternatively, the dose of the composition being administered is temporarily reduced or temporarily suspended for a certain length of time (i.e., a “drug holiday”). In some instances, the length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, 365 days, or 366 days. The dose reduction during a drug holiday is from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained.

In some embodiments, the amount of given phosphorylated gemcitabine derivative prodrug varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the amount of given phosphorylated gemcitabine derivative prodrug will typically be in the range of about 0.02 mg to about 5000 mg per day. In some embodiments, the amount of given phosphorylated gemcitabine derivative prodrug is in the range of about 1 mg to about 1500 mg per day. In some embodiments, the amount of given phosphorylated gemcitabine derivative prodrug is in the range of about 10 mg to about 1000 mg per day. The desired dose may conveniently be presented in a single dose or as divided doses administered simultaneously (or over a short period of time) or at appropriate intervals, for example as two, three, four or more sub-doses per day.

In some embodiments, the daily dosages appropriate for the phosphorylated gemcitabine derivative prodrug described herein are from about 0.01 mg/kg to about 100 mg/kg. In one embodiment, the daily dosages are from about 0.1 mg/kg to about 10 mg/kg. An indicated daily dosage in the larger mammal, including, but not limited to, humans, is in the range from about 0.5 mg to about 1000 mg, conveniently administered in a single dose or in divided doses. Suitable unit dosage forms for oral administration include from about 1 to about 500 mg active ingredient. In one embodiment, the unit dosage is about 1 mg, about 5 mg, about, 10 mg, about 20 mg, about 50 mg, about 100 mg, about 200 mg, about 250 mg, about 400 mg, or about 500 mg.

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Treatment of Cancer

In some embodiments, described herein is a method of treating cancer in a subject in need thereof, comprising administering to the subject a pharmaceutical composition comprising a phosphorylated gemcitabine derivative prodrug, or pharmaceutically acceptable salt thereof, as described herein.

In some embodiments, the cancer is pancreatic cancer, lung cancer, breast cancer, bladder cancer, biliary tract cancer, urethral cancer, testicular cancer, colorectal cancer, head and neck cancer, or ovarian cancer. In some embodiments, the lung cancer is non small cell lung cancer. In some embodiments, the breast cancer is metastatic breast cancer. In some embodiments, the biliary tract cancer is cholangiocarcinoma.

Some embodiments provide the method of treating pancreatic cancer, wherein the pancreatic cancer is selected from an epitheliod carcinoma in the pancreatic duct tissue or an adenocarcinoma in a pancreatic duct.

Provided herein is a method of treating cancer in a patient in need thereof comprising administering to the patient a pharmaceutical composition comprising a compound of Formula (I), (II), and (IIa), or a compound disclosed in Table 1, or pharmaceutically acceptable salt thereof, and a pharmaceutically acceptable excipient. In some embodiments, the cancer is pancreatic cancer, lung cancer, breast cancer, bladder cancer, biliary tract cancer, urethral cancer, testicular cancer, colorectal cancer, head and neck cancer, or ovarian cancer. In some embodiments, the lung cancer is non small cell lung cancer. In some embodiments, the breast cancer is metastatic breast cancer. In some embodiments, the biliary tract cancer is cholangiocarcinoma. Some embodiments provide the method of treating pancreatic cancer, wherein the pancreatic cancer is selected from an epitheliod carcinoma in the pancreatic duct tissue or an adenocarcinoma in a pancreatic duct. Provided herein is the method wherein the pharmaceutical composition is administered orally. Provided herein is the method wherein the pharmaceutical composition is administered by injection. Provided herein is the method wherein the pharmaceutical composition is administered by intramuscular injection. Provided herein is the method wherein the intramuscular injection is a depot injection. Provided herein is the method wherein the depot injection provides a therapeutically effective concentration for a period of 2 days to 3 months. Provided herein is the method wherein the depot injection provides a therapeutically effective concentration for a period of about 2 days. Provided herein is the method wherein the depot injection provides a therapeutically effective concentration for a period of about 4 days. Provided herein is the method wherein the depot injection provides a therapeutically effective concentration for a period of about 7 days. Provided herein is the method wherein the depot injection provides a therapeutically effective concentration for a period of about 10 days. Provided herein is the method wherein the depot injection provides a therapeutically effective concentration for a period of about 1 week. Provided herein is the method wherein the depot injection provides a therapeutically effective concentration for a period of about 2 weeks. Provided herein is the method wherein the depot injection provides a therapeutically effective concentration for a period of about 3 weeks. Provided herein is the method wherein the depot injection provides a therapeutically effective concentration for a period of about 4 weeks. Provided herein is the method wherein the depot injection provides a therapeutically effective concentration for a period of about 5 weeks. Provided herein is the method wherein the depot injection provides a therapeutically effective concentration for a period of about 6 weeks. Provided herein is the method wherein the depot injection provides a therapeutically effective concentration for a period of about 1 month. Provided herein is the method wherein the depot injection provides a therapeutically effective concentration for a period of about 2 months. Provided herein is the method wherein the depot injection provides a therapeutically effective concentration for a period of about 3 months.

Other embodiments and uses will be apparent to one skilled in the art in light of the present disclosures. The following examples are provided merely as illustrative of various embodiments and shall not be construed to limit the invention in any way.

### EXAMPLES

I. Chemical Synthesis

In some embodiments, the phosphorylated gemcitabine derivative prodrug compounds disclosed herein are synthesized according to the following examples.

General Scheme 1 for the synthesis of mono-phosphorylated gemcitabine derivative prodrug compounds.

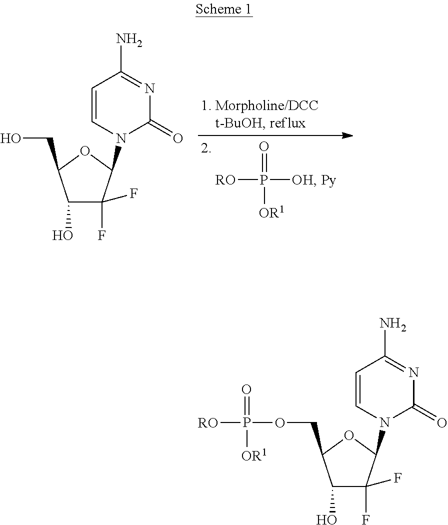

II. Biological Evaluation

### Example 1

### Plasma Stability Assay

Determining plasma stability of the test compounds is performed using HPLC-MS. Incubations are carried out in 96-well polypropylene plates in 5 aliquots of 70 μL each (one for each time point). Test compounds (10 μM, final solvent concentration 1%) are incubated at 37° C. Five time points over 120 minutes are analyzed (0, 20, 40, 60 and 120 min). All incubations are performed in duplicates. The samples are analyzed by HPLC-MS (API3000, AB Sciex). The percentage of parent compound remaining after incubation in plasma is plotted versus incubation time, and plasma half-life (T½) is calculated from the obtained curve.

III. Preparation of Pharmaceutical Dosage Forms

### Example 1

### Oral Capsule

The active ingredient is a compound of Table 1 or 2, or a pharmaceutically acceptable salt thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

### Example 2

### Solution for Injection

The active ingredient is a compound of Table 1 or 2, or a pharmaceutically acceptable salt thereof, and is formulated as a solution in sesame oil at a concentration of 50 mg-eq/mL.

1. A compound, or pharmaceutically acceptable salt thereof, having the structure of Formula (I): 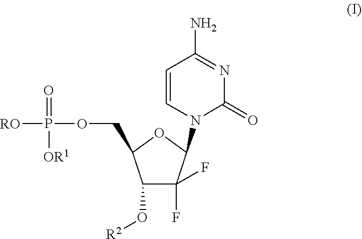
wherein
R is selected from fatty acid, glycerolipid, glycerophospholipids, sphingolipids, sterol-lipids, prenol lipids, saccharolipids and polyketides;
R¹ is selected from hydrogen, fatty acid, glycerolipid, glycerophospholipids, sphingolipids, sterol-lipids, prenol lipids, saccharolipids and polyketides; and
R² is selected from hydrogen, fatty acid, glycerolipid, glycerophospholipids, sphingolipids, sterol-lipids, prenol lipids, saccharolipids and polyketides.

2. The compound of claim 1, or pharmaceutically acceptable salt thereof, wherein R¹ is hydrogen or a fatty acid, and R is a fatty acid.

3. (canceled)

4. The compound of claim 1, or pharmaceutically acceptable salt thereof, wherein the fatty acid is independently selected from the group consisting of a saturated, monounsaturated, polyunsaturated fatty acid, a C2-C26 fatty acid, docosahexaenoic acid, eicosapentaenoic acid, docosahexaenoic acid, eicosapentaenoic acid, oleic acid, stearic acid, (9Z,12Z)-octadeca-9,12-dienoic acid, (Z)-docos-13-enoic acid, docosanoic acid, (E)-octadec-9-enoic acid, icosanoic acid, (9Z,12Z,15Z)-octadeca-9,12,15-trienoic acid, palmitic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, and hexacosanoic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, and eicosatrienoic acid, stearidonic acid, arachidonic acid, eicosatetraenoic acid, and adrenic acid.

5-12. (canceled)

13. A compound, or pharmaceutically acceptable salt thereof, having the structure of Formula (II): 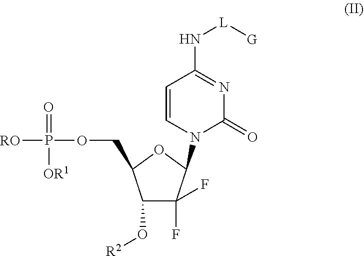
wherein
R is selected from hydrogen, fatty acid, glycerolipid, glycerophospholipids, sphingolipids, sterol-lipids, prenol lipids, saccharolipids and polyketides;
R¹ is selected from hydrogen, fatty acid, glycerolipid, glycerophospholipids, sphingolipids, sterol-lipids, prenol lipids, saccharolipids and polyketides;
R² is selected from hydrogen, fatty acid, glycerolipid, glycerophospholipids, sphingolipids, sterol-lipids, prenol lipids, saccharolipids and polyketides;
L is a linker selected from an alkylene amide group, an alkylene ester group, an alkylene carbamate group, a disulfide group, a phosphodiester group, and a phosphoramidate group; and
G is a cytotoxic chemotherapy agent.

14. The compound of claim 13, or pharmaceutically acceptable salt thereof, wherein the cytotoxic chemotherapy agent is selected from abiraterone, afatinib, axitinib, azacitidine, bortezomib, cabazitaxel, cabozantinib, capecitabine, carfilzomib, ceritinib, crizotinib, cyclophosphamide, cytarabine, dabrafenib, dactinomycin, dasatinib, daunorubicin, decarbazine, decitabine, docetaxel, doxorubicin, epirubicin, erlotinib, etoposide, everrolimus, floxuridine, gefitinib, ibrutinib, idarubicin, idelalisib, lapatinib, lenvatinib, leucovorin, methotrexate, mitomycin, olaparib, palbociclib, pazopanib, ponatinib, pralatrexate, prednisone, regorafenib, ruxolitinib, sorafenib, streptozocin, sunitinib, thalidomide, topotecan, vemurafenib, vincristine, vinorelbine, and zoledronic acid.

15. The compound of claim 13, or pharmaceutically acceptable salt thereof, wherein L is an alkylene carbamate group.

16. The compound of claim 13, or pharmaceutically acceptable salt thereof, wherein the compound has the structure of Formula (IIa): 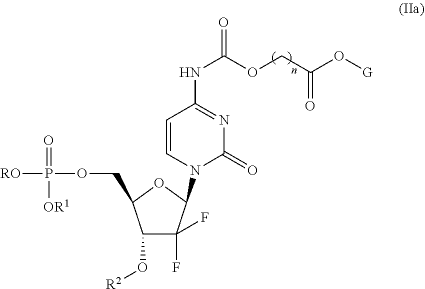
wherein n is 1 to 6.

17. The compound of claim 13, or pharmaceutically acceptable salt thereof, wherein R¹ is hydrogen or a fatty acid, and R is a fatty acid.

18 (canceled)

19. The compound of claim 17, or pharmaceutically acceptable salt thereof, wherein each fatty acid is independently selected from the group consisting of a saturated, monounsaturated, polyunsaturated fatty acid, a C2-C26 fatty acid, docosahexaenoic acid, eicosapentaenoic acid, docosahexaenoic acid, eicosapentaenoic acid, oleic acid, stearic acid, (9Z,12Z)-octadeca-9,12-dienoic acid, (Z)-docos-13-enoic acid, docosanoic acid, (E)-octadec-9-enoic acid, icosanoic acid, (9Z,12Z,15Z)-octadeca-9,12,15-trienoic acid, palmitic acid, butanoic acid, pentanoic acid, hexanoic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, eicosanoic acid, heneicosanoic acid, docosanoic acid, tricosanoic acid, tetracosanoic acid, pentacosanoic acid, hexacosanoic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, and adrenic acid.

20-27. (canceled)

28. A pharmaceutical composition comprising a compound of claim 1, or pharmaceutically acceptable salt thereof, and a pharmaceutically acceptable excipient.

29. The pharmaceutical composition of claim 28, wherein the composition is suitable for administration by injection.

30. The pharmaceutical composition of claim 29, wherein the composition is formulated as an albumin-coated nanoparticle or a lipid-based nanoparticle.

31. (canceled)

32. The pharmaceutical composition of claim 29, wherein the compound is incorporated in the lipid bilayer of a liposomal nanoparticle, inside a liposomal nanoparticle, or in a polymeric nanoparticle.

33-34. (canceled)

35. The pharmaceutical composition of claim 29, wherein the compound is incorporated in a polymeric or lipid nanoparticle that is coated with a poly ethylene glycol polymer, and the poly ethylene glycol polymer molecular weight of polyethylene glycol is 500 to 5000 daltons.

36-40. (canceled)

41. A compound comprising a gemcitabine derivative prodrug, wherein the prodrug comprises a modified mono-phosphate form of gemcitabine.

42. The compound of claim 41, wherein the compound does not require further phosphorylation to be in an active form.

43. The compound of claim 41, wherein the compound is less susceptible to resistance than gemcitabine.

44. The compound of claim 41, wherein the resistance comprises resistance caused by a cell's inability to phosphorylate gemcitabine.

45. The compound of claim 41, wherein the compound exhibits increased uptake by cells as compared to gemcitabine.

46. The compound of claim 41, wherein the compound enters a cell by a different mechanism than gemcitabine.

47-60. (canceled)
